(19) United States
(12) Patent Application Publication
(10) Pub. No.: US 2023/0302297 A1
(43) Pub. Date: Sep. 28, 2023

(54) PATIENT IMAGING FOR DYNAMIC ONLINE ADAPTIVE RADIOTHERAPY

(71) Applicant: Elekta Limited, Montreal (CA)

(72) Inventors: Martin Emile Lachaine, Montreal (CA); Tony Falco, La Prairie (CA)

(21) Appl. No.: 18/041,871

(22) PCT Filed: Aug. 17, 2021
(86) PCT No.: PCT/CA2021/051133

§ 371 Date: Feb. 16, 2023

Related U.S. Application Data
(60) Provisional application No. 63/066,552, filed on Aug. 17, 2020.
(60) Provisional application No. 63/066,542, filed on Aug. 17, 2020.

(51) Int. Cl. A61N 5/10

(52) U.S. Cl. CPC A61N 5/1037; A61N 5/1049; A61N 5/1039; A61N 5/1038

(57) ABSTRACT

Techniques are described that use surface camera imaging data combined with other information to describe how a patient is moving in 4D. Intrabody imaging data, such as from CT images, and surface camera imaging data, such as from surface imaging cameras, can be acquired. A system can generate a model relating the intrabody imaging data having a three-dimensional (3D) patient representation to a two-dimensional (2D) surface patient representation. During a particular treatment fraction session, the system can obtain surface camera imaging data and use the surface camera imaging data and the model to calculate a 3D patient representation during the particular treatment fraction session. In this manner, surface camera imaging data can drive the model to provide motion management during (or before or after) a treatment session so that the 3D state of a patient is known at any given moment during (or before or after) a treatment session.

### CLAIM OF PRIORITY

This application claims the benefit of priority of U.S. Provisional Patent Application Ser. No. 63/066,542, titled “PATIENT IMAGING FOR ONLINE ADAPTIVE RADIOTHERAPY” to Martin Emile Lachaine et al., filed on Aug. 17, 2020, and U.S. Provisional Patent Application Ser. No. 63/066,552, titled “PATIENT IMAGING FOR DYNAMIC ONLINE ADAPTIVE RADIOTHERAPY” to Martin Emile Lachaine et al., filed on Aug. 17, 2020, the entire contents of each being incorporated herein by reference.

### TECHNICAL FIELD

Embodiments of the present disclosure pertain generally to radiotherapy treatment sessions and specifically to imaging techniques.

### BACKGROUND

Radiation therapy (or “radiotherapy”) can be used to treat cancers or other ailments in mammalian (e.g., human and animal) tissue. One such radiotherapy technique involves irradiation with a Gamma Knife, whereby a patient is irradiated by a large number of low-intensity gamma ray beams that converge with high intensity and high precision at a target (e.g., a tumor). In another embodiment, radiotherapy is provided using a linear accelerator, whereby a tumor is irradiated by high-energy particles (e.g., electrons, protons, ions, high-energy photons, and the like). The placement and dose of the radiation beam must be accurately controlled to ensure the tumor receives the prescribed radiation, and the placement of the beam should be such as to minimize damage to the surrounding healthy tissue, often called the organ(s) at risk (OARs). Radiation is termed “prescribed” because a physician orders a predefined amount of radiation to the tumor and surrounding organs similar to a prescription for medicine. Generally, ionizing radiation in the form of a collimated beam is directed from an external radiation source toward a patient.

A specified or selectable beam energy can be used, such as for delivering a diagnostic energy level range or a therapeutic energy level range. Modulation of a radiation beam can be provided by one or more attenuators or collimators (e.g., a multi-leaf collimator (MLC)). The intensity and shape of the radiation beam can be adjusted by collimation to avoid damaging healthy tissue (e.g., OARs) adjacent to the targeted tissue by conforming the projected beam to a profile of the targeted tissue.

The treatment planning procedure may include using a three-dimensional (3D) image of the patient to identify a target region (e.g., the tumor) and to identify critical organs near the tumor. Creation of a treatment plan can be a time-consuming process where a planner tries to comply with various treatment objectives or constraints (e.g., dose volume histogram (DVH), overlap volume histogram (OVH)), taking into account their individual importance (e.g., weighting) in order to produce a treatment plan that is clinically acceptable. This task can be a time-consuming trial-and-error process that is complicated by the various OARs because as the number of OARs increases (e.g., up to thirteen for a head-and-neck treatment), so does the complexity of the process. OARs distant from a tumor may be easily spared from radiation, while OARs close to or overlapping a target tumor may be difficult to spare.

Traditionally, for each patient, the initial treatment plan can be generated in an “offline” manner. The treatment plan can be developed well before radiation therapy is delivered, such as using one or more medical imaging techniques. Imaging information can include, for example, images from X-rays, computed tomography (CT), nuclear magnetic resonance (MR), positron emission tomography (PET), single-photon emission computed tomography (SPECT), or ultrasound. A health care provider, such as a physician, may use 3D imaging information indicative of the patient anatomy to identify one or more target tumors along with the OARs near the tumor(s). The health care provider can delineate the target tumor that is to receive a prescribed radiation dose using a manual technique, and the health care provider can similarly delineate nearby tissue, such as organs, at risk of damage from the radiation treatment. Alternatively, or additionally, an automated tool (e.g., ABAS provided by Elekta AB, Sweden) can be used to assist in identifying or delineating the target tumor and organs at risk. A radiation therapy treatment plan (“treatment plan”) can then be created using an optimization technique based on clinical and dosimetric objectives and constraints (e.g., the maximum, minimum, and fraction of dose of radiation to a fraction of the tumor volume (“95% of target shall receive no less than 100% of prescribed dose”), and like measures for the critical organs). The optimized plan is comprised of numerical parameters that specify the direction, cross-sectional shape, and intensity of each radiation beam.

The treatment plan can then be later executed by positioning the patient in the treatment machine and delivering the prescribed radiation therapy directed by the optimized plan parameters. The radiation therapy treatment plan can include dose “fractioning,” whereby a sequence of radiation treatments is provided over a predetermined period of time (e.g., 30-45 daily fractions), with each treatment including a specified fraction of a total prescribed dose. However, during treatment, the position of the patient and the position of the target tumor in relation to the treatment machine (e.g., linear accelerator—“linac”) is very important in order to ensure the target tumor and not healthy tissue is irradiated.

Since most patients receive more than one fraction of radiation as part of a course of therapy, and because the anatomy may change (deform) between these fractions, it is not straightforward to sum the doses delivered during the individual fractions so the physician can accurately gauge how the treatment is proceeding relative to the original intent as defined by the prescription.

### OVERVIEW

This disclosure describes techniques that use surface camera imaging data combined with other a priori information to describe how a patient is moving in 4D. As described in more detail below, intrabody imaging data, such as from CT images, and surface camera imaging data, such as from surface imaging cameras, can be acquired. A system can generate a model relating the intrabody imaging data having a three-dimensional (3D) patient representation to a two-dimensional (2D) surface patient representation. During a particular treatment fraction session, the system can obtain surface camera imaging data and use the surface camera imaging data and the model to calculate a 3D patient representation during the particular treatment fraction session. In this manner, surface camera imaging data can drive the model to provide motion management during (or before or after) a treatment session so that the 3D state of a patient is known at any given moment during (or before or after) a treatment session.

In some aspects, this disclosure is directed to a computer-implemented radiation treatment planning method, the method comprising: obtaining intrabody imaging data and surface imaging data for determining a radiation treatment plan including a plurality of radiation treatment fraction sessions; using the intrabody imaging data and the surface imaging data, generating a model relating 1) the intrabody imaging data having a three-dimensional (3D) patient representation to 2) a two-dimensional (2D) surface patient representation; obtaining surface camera imaging data during a particular treatment fraction session; and using the surface camera imaging data obtained during the particular treatment fraction session and the model, calculating a 3D patient representation during the particular treatment fraction session.

In some aspects, this disclosure is directed to a radiation treatment system configured to: obtain intrabody imaging data and surface imaging data for determining a radiation treatment plan including a plurality of radiation treatment fraction sessions; use the intrabody imaging data and the surface imaging data to generate a model relating 1) the intrabody imaging data having a three-dimensional (3D) patient representation to 2) a two-dimensional (2D) surface patient representation; obtain surface camera imaging data during a particular treatment fraction session; and use the surface camera imaging data obtained during the particular treatment fraction session and the model to calculate a 3D patient representation during the particular treatment fraction session.

In some aspects, this disclosure is directed to a tangible or non-tangible computer readable medium encoded with instructions that, when executed by a processor, cause the processor to: obtain intrabody imaging data and surface imaging data for determining a radiation treatment plan including a plurality of radiation treatment fraction sessions; use the intrabody imaging data and the surface imaging data to generate a model relating 1) the intrabody imaging data having a three-dimensional (3D) patient representation to 2) a two-dimensional (2D) surface patient representation; obtain surface camera imaging data during a particular treatment fraction session; and use the surface camera imaging data obtained during the particular treatment fraction session and the model to calculate a 3D patient representation during the particular treatment fraction session.

In some aspects, this disclosure is directed to a computer-implemented radiation treatment planning method, the method comprising: obtaining pre-treatment CT imaging data for determining a radiation treatment plan including a plurality of radiation treatment fraction sessions; obtaining CBCT imaging data during a treatment fraction session; obtaining surface camera imaging data during the treatment fraction session; and generating synthetic CT (sCT) imaging data from the CBCT imaging data and the surface camera imaging data.

In some aspects, this disclosure is directed to a radiation treatment system configured to: obtain pre-treatment CT imaging data for determining a radiation treatment plan including a plurality of radiation treatment fraction sessions; obtain CBCT imaging data during a treatment fraction session; obtain surface camera imaging data during the treatment fraction session; and generate synthetic CT (sCT) imaging data from the CBCT imaging data and the surface camera imaging data.

In some aspects, this disclosure is directed to a tangible or non-tangible computer readable medium encoded with instructions that, when executed by a processor, cause the processor to: obtain pre-treatment CT imaging data for determining a radiation treatment plan including a plurality of radiation treatment fraction sessions; obtain CBCT imaging data during a treatment fraction session; obtain surface camera imaging data during the treatment fraction session; and generate synthetic CT (sCT) imaging data from the CBCT imaging data and the surface camera imaging data.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example but not by way of limitation, various embodiments discussed in the present document.

FIG. 1 illustrates an example of a radiotherapy system, according to some embodiments of the present disclosure.

FIG. 2A illustrates an example a radiation therapy system that can include radiation therapy output configured to provide a therapy beam, according to some embodiments of the present disclosure.

FIG. 2B illustrates an example of a system including a combined radiation therapy system and an imaging system, such as a cone beam computed tomography (CBCT) imaging system, according to some embodiments of the present disclosure.

FIG. 3 illustrates a partially cut-away view of an example system including a combined radiation therapy system and an imaging system, such as a nuclear MR imaging (MRI) system, according to some embodiments of the present disclosure.

FIGS. 4A and 4B depict the differences between an example MRI image and a corresponding CT image, respectively, according to some embodiments of the present disclosure.

FIG. 5 illustrates an example of a collimator configuration for shaping, directing, or modulating an intensity of a radiation therapy beam, according to some embodiments of the present disclosure.

FIG. 6 illustrates an example of a Gamma Knife radiation therapy system, according to some embodiments of the present disclosure.

FIG. 7 illustrates a flow diagram of an example of a computer-assisted radiation treatment planning and treatment delivery method, according to some embodiments of the present disclosure.

FIG. 8A depicts a conceptualized diagram of a planning CT image.

FIG. 8B depicts a conceptualized diagram of a limited FOV CBCT acquired during a radiotherapy fraction as well as a partial external contour measured with the surface camera.

FIG. 9 is a conceptualized diagram illustrating how the surface points from the time of treatment planning in FIG. 8A relate to the surface points at the time of treatment in FIG. 8B.

FIG. 10 illustrates a flow diagram of another example of a computer-assisted radiation treatment planning and treatment delivery method 1000, according to some embodiments of the present disclosure.

FIG. 11 illustrates an example of a block diagram of a machine on which one or more of the methods as discussed herein can be implemented.

### DETAILED DESCRIPTION

In the following detailed description, reference is made to the accompanying drawings which form a part hereof, and which is shown by way of illustration-specific embodiments in which the present disclosure may be practiced. These embodiments, which are also referred to herein as “examples,” are described in sufficient detail to enable those skilled in the art to practice the disclosure, and it is to be understood that the embodiments may be combined, or that other embodiments may be utilized, and that structural, logical and electrical changes may be made without departing from the scope of the present disclosure. The following detailed description is, therefore, not be taken in a limiting sense, and the scope of the present disclosure is defined by the appended claims and their equivalents.

In an approach to non-adaptive radiotherapy, a diagnostic quality simulation computed tomography (CT) dataset can be acquired as a primary imaging dataset for radiation treatment planning. A simulation CT can provide two key inputs for generating a radiation treatment plan: a structure set and an Electron Density (ED) map. The structure set can include the target, organs-at-risk, and external contour, as well as certain other structures of interest. In some cases, complementary secondary datasets such as MRI or PET can be used, such as to assist with structure definition. The ED map can be assigned on a voxel-by-voxel basis by a calibration curve that relates CT numbers to ED values, or average ED values may be assigned to corresponding individual structures.

The radiation treatment planning can generate a patient prescription. The patient prescription can include specification of a number of individual treatment “fractions” in which the course of radiation treatment is to be delivered over a course of time that may include inpatient or outpatient treatment sessions. In one approach to non-adaptive radiotherapy, it is assumed that the patient anatomy does not deform significantly from fraction-to-fraction, and so the same treatment plan is delivered at each fraction. Images such as cone-beam CT (CBCT) may be acquired during such fraction treatment sessions, but they are only used to improve patient alignment for a radiation treatment session, not to modify the treatment plan, such as in view of a change in patient anatomy.

Online adaptive radiotherapy can adapt the treatment plan, such as to better conform to the deformable patient anatomy for each fraction. If an integrated CT scanner is available in the treatment room with the radiation delivery device, then the same quality of imaging information is available as was available for generating the initial treatment plan. The new CT information can be used to either generate a new structure set from scratch, or the original structures can be deformed to match the new CT. ED values can be directly determined from the new CT. With this information, a new treatment plan can be generated that conforms to the patient's anatomy as presented during a particular treatment fraction.

Most linear accelerators, however, have integrated cone-beam CT (CBCT) functionality instead of diagnostic CT. In some cases, the CBCT can be used directly for treatment planning, but the CBCT image quality is generally inferior to diagnostic CT, e.g., exhibiting lower contrast and exhibiting artifacts. For this reason, some approaches to adaptive workflows can generate a synthetic CT (sCT) from the CBCT image, which synthetically attempts to replicate the acquisition of a new diagnostic CT.

One method that can be used to generate a sCT is to first calculate a deformation vector field (DVF), which relates each point in the planning CT to a new position in the treatment coordinate system. Once the DVF has been determined, is can be used to deform the planning CT to generate the sCT. The treatment planning structure set can then also be propagated to the time of treatment using this same DVF.

A DVF between two images may be generated such as using an iterative optimization process, such as one that finds the DVF which, when applied to the second image, results in the highest similarity between the first image and deformed second image. Regularization can be used such as to help ensure that the DVF is as physically realistic as possible. Additional constraints or limitations may also be enforced during the optimization. For example, paired structure sets on each image may be used to constrain the DVF such that, when deformed by the DVF, the deformed structures superimpose on each other, e.g., exactly. Other methods of generating sCT have can be used, such as using deep learning.

Although static adaptive radiotherapy can be useful for adapting to deformations that occur from fraction-to-fraction (interfractional motion, e.g., between treatment fraction sessions), they do not account for deformations that occur during the treatment itself (intrafractional motion). Dynamic adaptive radiotherapy can adapt the treatment plan to better conform to the patient's current anatomy while the beam is on. This can include accommodating any changes in the patient's positioning during such time. For target sites that are strongly affected by respiratory motion, for example, it may not be possible to obtain a full 3D CT or CBCT with sufficient speed (e.g., one 3D image per <100 ms, or at most per 500 ms if a prediction algorithm is used to compensate for imaging lag). Thus, it is desirable to provide techniques that can be used to generate the 3D images from fast partial information.

For respiratory targets, “4D” imaging can be used instead of 3D imaging, such as for both CT and CBCT modalities. These 4D images are not really 4D in the sense of 3D+time. Rather, they assume that each respiratory cycle is the same, which allows the reconstruction algorithms to bin projection information from different respiratory cycles together, such as according to respiratory cycle phase. A 3D image can be reconstructed from each bin, leading to a 4D image sequence corresponding to a representative respiratory cycle. The 4D information can then be collapsed to a representative respiratory phase for planning and treatment, as well as to help aid in designing margins that adequately encompass respiratory motion. The 4D image information cannot, however, be used to directly calculate the instantaneous patient 3D image at a given point in time in radiotherapy. Breathing cycles can vary significantly from one breath to the other, and 4D imaging is insufficient for cases in which a clinician wishes to dynamically adapt to each breathing cycle.

Examples of methods to calculate real-time evolving 3D images from intrafractional imaging data; either using 2D MR slices on an MR linac, or kV imaging, optionally in combination with surface camera data, on a conventional linac are described in US2020129780, US20170360325, US20200129784, US20200160972, and U.S. Provisional Application No. 62/991,356, (Attorney Docket No. 4186.139PRV; Client Ref. No. 19US11PROV), filed Mar. 18, 2020, each of which is incorporated by reference herein, its entirety.

These approaches, when applied to a linac, rely primarily on kV imaging during the treatment itself. Some issues with a kV imaging implementation are the following:
a) kV images add radiation dose to the patient. As mentioned in US2020129780, this can be mitigated by also acquiring surface data in parallel, enabling a lower frequency of kV imaging, which in turn reduces dose, but not completely;
b) kV image quality can be relatively poor on individual projections and may not always contain sufficient information;
c) The above-mentioned approaches (except for U.S. Provisional Application No. 62/991,356) operate by first building a model based on 4D images, and then estimating the parameters of that model based on the real-time kV images. 4D CBCT may suffer from limited field of view (FOV) and so may be insufficient to generate a model that requires the full 3D patient anatomy. A planning 4D CT could be used to build the model instead, but the large time difference between simulation and treatment would likely cause the model to not be reliably applicable; and
d) Some linac configurations may not include kV imaging, for example they may integrate a diagnostic quality CT scanner instead.

Although surface cameras have been mentioned in US2020129780, such as in order to reduce radiation dose, this document describes an approach in which one or more surface cameras are the primary, and in some cases the only, technology to drive calculation of the instantaneous 3D image of the patient for dynamic adaptive radiotherapy.

Surface information can be used as a surrogate for internal anatomy. However, it is generally only reliable for relatively short periods of time. Furthermore, even if it can be used to determine the 3D location of a target centroid, that does not necessarily mean that it can be used to generate a full 3D image of the patient that is needed for dynamic adaptive radiotherapy.

This disclosure describes techniques that use surface camera imaging data combined with other a priori information to describe how a patient is moving in 4D. As described in more detail below, intrabody imaging data, such as from CT images, and surface camera imaging data, such as from surface imaging cameras, can be acquired. A system can generate a model relating the intrabody imaging data having a three-dimensional (3D) patient representation to a two-dimensional (2D) surface patient representation. During a particular treatment fraction session, the system can obtain surface camera imaging data and use the surface camera imaging data and the model to calculate a 3D patient representation during the particular treatment fraction session. In this manner, surface camera imaging data can drive the model to provide motion management during (or before or after) a treatment session so that the 3D state of a patient is known at any given moment during (or before or after) a treatment session. For example, surface camera imaging data can be used to confirm proper patient positioning and verify a treatment plan before a treatment session. In addition, in some examples, surface camera imaging data can be used after a treatment session to assess the effect of the treatment based on any movement encountered during the treatment session.

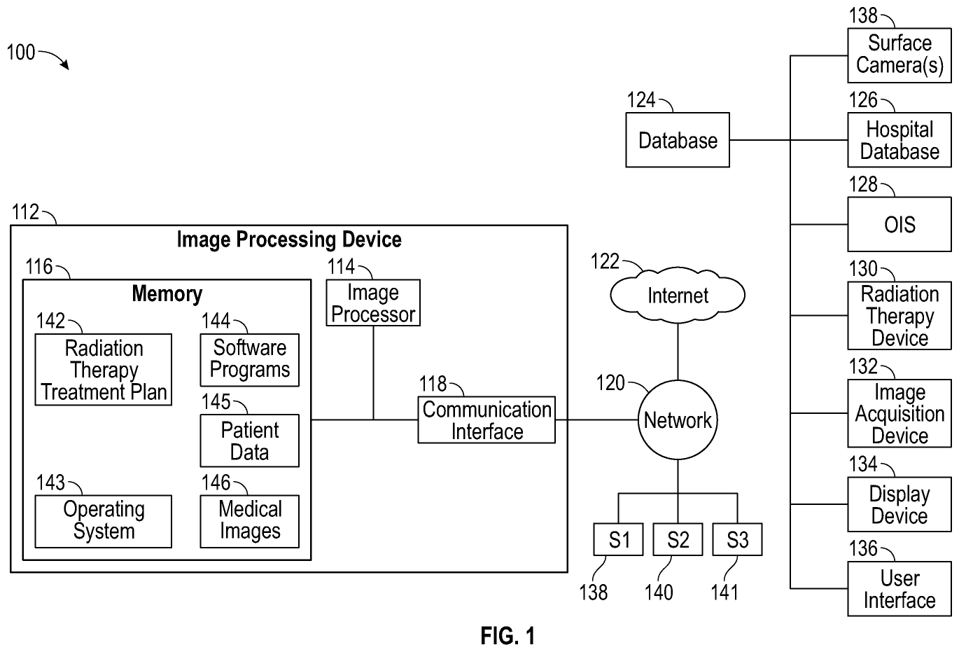

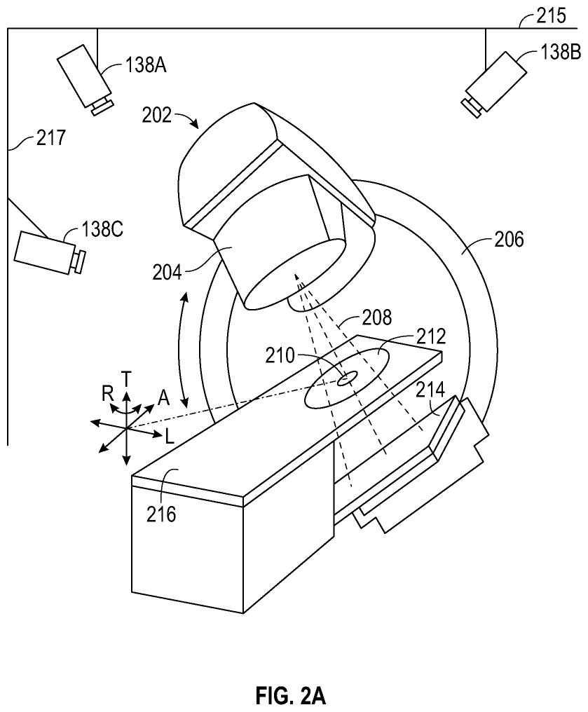

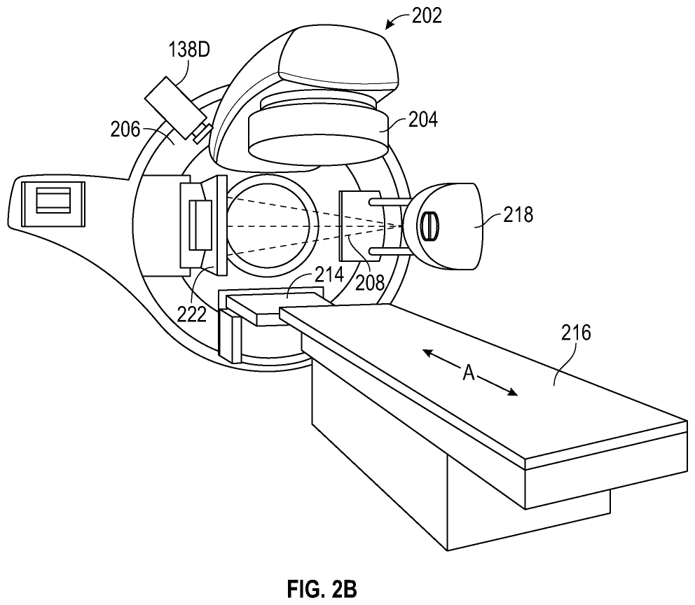

FIG. 1 illustrates an example of a radiotherapy system 100 for providing radiation therapy to a patient. The radiotherapy system 100 includes an image processing device 112. The image processing device 112 may be connected to a network 120. The network 120 may be connected to the Internet 122. The network 120 can connect the image processing device 112 with one or more of a database 124, a hospital database 126, an oncology information system (OIS) 128, a radiation therapy device 130, an image acquisition device 132, a display device 134, a user interface 136, and one or more surface cameras 138, such as surface cameras 138A-138C in FIG. 2A and/or surface camera 138D in FIG. 2B. Examples of surface cameras 138 can include those manufactured by C-Rad, VisionRT, and Varian HumediQ. The surface camera(s) 138 (e.g., one or more 2D or 3D cameras) can be used to acquire real-time images of the surface of a patient's body (e.g., the patient's skin) while medical images are being acquired. Because the surface imaging is taken at the same time as the medical imaging, the surface imaging can provide a more accurate definition of the location of the boundaries of the patient's body while the medical imaging was taken. The image processing device 112 can be configured to generate radiation therapy treatment plans 142 to be used by the radiation therapy device 130.

The image processing device 112 may include a memory device 116, an image processor 114, and a communication interface 118. The memory device 116 may store computer-executable instructions, such as an operating system 143, radiation therapy treatment plans 142 (e.g., original treatment plans, adapted treatment plans and the like), software programs 144 (e.g., artificial intelligence, deep learning, neural networks, radiotherapy treatment plan software), and any other computer-executable instructions to be executed by the image processor 114.

In one embodiment, the software programs 144 may convert medical images of one format (e.g., MRI) to another format (e.g., CT) by producing synthetic images, such as pseudo-CT images. For instance, the software programs 144 may include image processing programs to train a predictive model for converting a medical image 146 in one modality (e.g., an MRI image) into a synthetic image of a different modality (e.g., a pseudo CT image); alternatively, the trained predictive model may convert a CT image into an MRI image.

In another embodiment, the software programs 144 may register the patient image (e.g., a CT image or an MR image) with that patient's dose distribution (also represented as an image) so that corresponding image voxels and dose voxels are associated appropriately by the network.

In yet another embodiment, the software programs 144 may substitute functions of the patient images or processed versions of the images that emphasize some aspect of the image information. Such functions might emphasize edges or differences in voxel textures, or any other structural aspect useful to neural network learning.

In another embodiment, the software programs 144 may substitute functions of the dose distribution that emphasize some aspect of the dose information. Such functions might emphasize steep gradients around the target or any other structural aspect useful to neural network learning. The memory device 116 may store data, including medical images 146, patient data 145, and other data required to create and implement a radiation therapy treatment plan 142.

In yet another embodiment, the software programs 144 may generate a structural estimate (e.g., a 3D model of the region of interest) using an iterative image reconstruction process. The structural estimate may be or include an X-ray attenuation map that represents a 3D model of a region of interest. The structural estimate may be used to estimate or simulate X-ray measurements to be compared with real X-ray measurements for updating the structural estimate. Specifically, the software programs 144 can access a current structural estimate of the region of interest and generate a first simulated X-ray measurement based on the current structural estimate of the region of interest.

A simulated X-ray measurement, as referred to herein, represents the expected output of an X-ray detector element when an X-ray source projects one or more X-ray beams through the region of interest towards the X-ray detector element. The simulated X-ray measurement can provide an expected image output that is to be received from the X-ray detector element.

The software programs 144 can receive a first real X-ray measurement from a CBCT system (or other CT imaging system, such as an enclosed gantry helical multi-slice CT with a curved detector or tomotherapy system) and generate an update to the current structural estimate of the region of interest as a function of the first simulated X-ray measurement and the first real X-ray measurement. A real X-ray measurement, as referred to herein, is an actual output that is received from a CBCT system (or other CT imaging system, such as an enclosed gantry helical multi-slice CT with a curved detector or tomotherapy system) that represents the amount of X-rays received by and detected by the X-ray detector along different directions, such as in an image form.

The update can be generated invariant on (independent of) the current structural estimate. The structural estimate can be used to control one or more radiotherapy treatment parameters by recalculating dose, adjusting one or more radiotherapy treatment machine parameters, or generating a display of the structural estimate on a graphical user interface.

In some embodiments, the software programs 144 generate the first simulated X-ray measurement based on the current structural estimate of the region of interest by applying the current structural estimate to a model that generates an expected output of a real X-ray measurement. In some implementations, the model that generates an expected output of a real X-ray measurement includes a modeling function representing beam hardening from a polyenergetic source. In some implementations, the modeling function that generates an expected output of a real X-ray measurement includes a machine learning technique that is trained to establish a relationship between a training real X-ray measurement and a training known simulated X-ray measurement. In some implementations, the modeling function that generates an expected output of a real X-ray measurement includes a linear model or a non-linear model that fits X-ray data to some nominal value comprising at least one of relative electron density, mass density, monoenergetic attenuation, proton stopping power, or bone mineral density.

As an example, the model generates the expected output of the real X-ray measurement, for a given measurement index i per number of X-ray detector elements in the CBCT system, in accordance with Equation 1:

zᵢ=bᵢ*exp(−[Ax]ᵢ)+rᵢ (1)

where b is an incident intensity of flood field, A is a system projection matrix describing a combination of each image pixel at each detector element, x is the current X-ray attenuation map, exp is an exponential function, and r is background noise including scatter. For example, the model applies the current structural estimate to Equation 1 as x to output the first simulated measurement z. The first simulated measurement can be generated for each of a plurality of X-ray detector elements that are in the CBCT system.

In an example, the value for r representing the noise including scatter can be re-estimated based on the current structural estimate at each iteration. For example, the noise including scatter for a given iteration can be applied to another machine learning technique or model including x (e.g., the current structural estimate) to generate an update and re-estimate the value of the noise including scatter.

In some embodiments, the software programs 144 compute the update to the current structural estimate as a derivative of a statistical objective function with respect to the first simulated X-ray measurement. For example, the statistical objective function can be computed according to Equation 2:

A^{T}(y./(b.*exp(−Ax)+r)−1) (2)

where A is a system projection matrix describing a combination of each image pixel at each detector element, A^{T} is a transpose of the system projection matrix, y is the first real X-ray measurement, b is an incident intensity of flood field, x is the current X-ray attenuation map, exp is an exponential function, and r is background noise including scatter. The update to the current structural estimate represents a perturbation to the current structural estimate. Namely, the update is linearly projected to the current structural estimate into an image space to form a perturbation. The perturbation is scaled by a scalar for stability and the scaled perturbation is subtracted from the current structural estimate to generate an updated structural estimate.

In some implementations, after the structural estimate is updated, at least one of regularization, momentum or denoising is applied to the updated structural estimate to improve the image quality of the 3D model represented by the structural estimate.

In some embodiments, the computation of the update and the updating of the structural estimate is conditioned on and performed in response to a computation of a negative log-likelihood (NLL) function (e.g., a loss function). Specifically, the loss function is computed as a combination of the first simulated X-ray measurement and the first real X-ray measurement, such as in accordance with Equation 3:

NLL=sum(zᵢ−yᵢ*log(zᵢ)) (3)

which may represent a sum of the differences between each of the plurality of simulated and real measurements of the X-ray CBCT system. Namely, a plurality of simulated measurements each associated with a different X-ray detector can be generated in accordance with Equation 1. The corresponding real X-ray measurements can be received from the X-ray detector, such as from the image acquisition device 132. The differences between each simulated measurement and the corresponding real measurement can be computed and summed. If the sum of these differences is below a threshold (e.g., satisfies a stopping criterion), then the update to the structural estimate is not performed. On the other hand, in response to determining that the loss fails to satisfy the stopping criterion (e.g., if the sum of these differences is above the threshold), the update to the current structural estimate is performed.

In some cases, instead of or in addition to conditioning the performance of the update on the sum of the differences or the loss function defined by Equation 3, the stopping criterion (e.g., the condition for not performing the update) can include a difference between adjacent updates falling below a threshold. For example, the software programs 144 can access a previous update value(s) and can generate a new update value based on the currently generated simulated X-ray measurement. The software programs 144 can compute a difference between these two adjacent or subsequent update values to the structural estimate. The software programs 144 can compare this difference to a stopping threshold and if the difference is below the stopping threshold, the software programs 144 may avoid performing the update to the structural estimate. If the difference is above the stopping threshold, the software programs 144 can perform the update to the structural estimate, such as in accordance with Equation 2.

In some cases, instead of or in addition to conditioning the performance of the update on the sum of the differences or the loss function defined by Equation 3, the stopping criterion (e.g., the condition for not performing the update) can include a number of iterations falling below a maximum iteration value and/or an elapsed time falling below a maximum time limit. For example, software programs 144 can keep a running count of the total number of iterations performed in which at each iteration the update to the structural estimate is performed, such as in accordance with Equation 2. The software programs 144 can also start a timer when the first update is performed. If the total number of iterations is less than a maximum iteration value, then the software programs 144 can perform the update to the structural estimate; and if otherwise, then the update is not performed. As another example, if the total time since the first update was performed is less than the maximum time limit, then the software programs 144 can perform the update to the structural estimate; and if otherwise, then the update is not performed.

In some cases, instead of or in addition to conditioning the performance of the update on the sum of the differences or the loss function defined by Equation 3, the stopping criterion (e.g., the condition for not performing the update) can include input requesting termination and display of a result. For example, a user input can be received during the process of performing the iterative image reconstruction. The input may request display of the current structural estimate. In response, the software programs 144 stop performing the update and display the current structural estimate. User input can be received to resume the process of performing the iterative image reconstruction. In response, the software programs 144 perform the last generated update and continue performing additional updates until another stopping criterion is met or satisfied.

For example, the software programs 144 access an updated structural estimate of the region of interest. The software programs 144 generate a second simulated X-ray measurement based on the updated structural estimate of the region of interest, such as in accordance with Equation 1. The software programs 144 receive a second real X-ray measurement from the CBCT system. The software programs 144 generate a further update to the updated structural estimate of the region of interest as a function of the second simulated X-ray measurement and the second real X-ray measurement, such as in accordance with Equation 2.

In some examples, the software programs 144 reduce a number of iterations and efficiency of generating the updates to the structural estimate by portioning the X-ray measurements into measurement groups and only performing an update if a given real X-ray measurement falls into a group associated with updating the structural estimate. Specifically, the software programs 144 receive a plurality of real X-ray measurements. The X-ray measurements are partitioned into measurement groups, in which a first measurement group of the measurement groups corresponds to a group of X-ray measurements used to update the current X-ray attenuation map, and a second measurement group of the measurement groups corresponds to a group of X-ray measurements that is skipped from updating the current X-ray structural estimate. The software programs 144 determines that the first real X-ray measurement falls within the first measurement group and, in response, the software programs 144 perform the update to the current structural estimate.

In some cases, the measurement groups are divided by number or quantity of real X-ray measurements that are received. For example, a queue may be maintained in which the real X-ray measurements are input. The queue may include a number of entries. As each new real X-ray measurement is received, the X-ray measurement is input to the queue. When the number of entries in the queue reaches a specified threshold, the last received X-ray measurement or a subset of last received X-ray measurements are used to perform an update to the structural estimate, such as in accordance with Equation 2. For example, when the number of entries in the queue reaches the specified threshold, the software programs 144 use Equation 1 to generate a simulated set of X-ray measurements which are used in combination with the last received X-ray measurement or a subset of last received X-ray measurements to generate the update to the current structural estimate. After the current structural estimate is updated, the queue is flushed and the entries are deleted so that 0 entries remain in the queue. In this way, the next update is performed when the next set of real X-ray measurements fill up the entries in the queue to reach the specified threshold.

In some implementations, the current structural estimate is smoothed based on a collection of updates to the structural estimate. Specifically, the software programs 144 can add each computed new updated of the structural estimate to a collection of updates performed on the structural estimate. Each update in the collection corresponds to or is associated with a respective iteration of performing updates to the structural estimate. The software programs 144 can identify a pattern in the collected plurality of updates. The software programs 144 can adjust or perform a new update to the structural estimate based on the identified pattern in the collected plurality of updates. For example, the software programs 144 can generate a plurality of updates based on a plurality of previously generated simulated X-ray measurements and corresponding real X-ray measurements. Each of these updates is input to a collection and a machine learning model or heuristic is applied to the collection to identify a particular pattern. The software programs 144 can then generate a new simulated X-ray measurement and receive a new real X-ray measurement. The software programs 144 can compute a new update to the structural estimate based on the new simulated X-ray measurement and new real X-ray measurement, according to Equation 2. The software programs 144 can adjust the computed new update based on the particular pattern that has been identified in the collection of previous updates. This can smooth the updates by preventing performance of updates that are too large in a particular portion of the structural estimate.

In addition to the memory device 116 storing the software programs 144, it is contemplated that software programs 144 may be stored on a removable computer medium, such as a hard drive, a computer disk, a CD-ROM, a DVD, a HD, a Blu-Ray DVD, USB flash drive, a SD card, a memory stick, or any other suitable medium; and the software programs 144 when downloaded to image processing device 112 may be executed by image processor 114.

The processor 114 may be communicatively coupled to the memory device 116, and the processor 114 may be configured to execute computer-executable instructions stored thereon. The processor 114 may send or receive medical images 146 to memory device 116. For example, the processor 114 may receive medical images 146 from the image acquisition device 132 via the communication interface 118 and network 120 to be stored in memory device 116. The processor 114 may also send medical images 146 stored in memory device 116 via the communication interface 118 to the network 120 be either stored in database 124 or the hospital database 126.

Further, the processor 114 may utilize software programs 144 (e.g., a treatment planning software) along with the medical images 146 and patient data 145 to create the radiation therapy treatment plan 142. Medical images 146 may include information such as imaging data associated with a patient anatomical region, organ, or volume of interest segmentation data. Patient data 145 may include information such as (1) functional organ modeling data (e.g., serial versus parallel organs, appropriate dose response models, etc.); (2) radiation dosage data (e.g., DVH information); or (3) other clinical information about the patient and course of treatment (e.g., other surgeries, chemotherapy, previous radiotherapy, etc.).

In addition, the processor 114 may utilize software programs to generate intermediate data such as updated parameters to be used, for example, by a machine learning model, such as a neural network model; or generate intermediate 2D or 3D images, which may then subsequently be stored in memory device 116. The processor 114 may subsequently transmit the executable radiation therapy treatment plan 142 via the communication interface 118 to the network 120 to the radiation therapy device 130, where the radiation therapy plan will be used to treat a patient with radiation. In addition, the processor 114 may execute software programs 144 to implement functions such as image conversion, image segmentation, deep learning, neural networks, and artificial intelligence. For instance, the processor 114 may execute software programs 144 that train or contour a medical image; such software programs 144 when executed may train a boundary detector or utilize a shape dictionary.

The processor 114 may be a processing device, including one or more general-purpose processing devices such as a microprocessor, a central processing unit (CPU), a graphics processing unit (GPU), an accelerated processing unit (APU), or the like. More particularly, the processor 114 may be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processor 114 may also be implemented by one or more special-purpose processing devices such as an application-specific integrated circuit (ASIC), a field programmable gate array (FPGA), a digital signal processor (DSP), a System on a Chip (SoC), or the like. As would be appreciated by those skilled in the art, in some embodiments, the processor 114 may be a special-purpose processor rather than a general-purpose processor. The processor 114 may include one or more known processing devices, such as a microprocessor from the Pentium™, Core™, Xeon™, or Itanium® family manufactured by Intel™, the Turion™, Athlon™, Sempron™, Opteron™, FX™, Phenom™ family manufactured by AMD™, or any of various processors manufactured by Sun Microsystems. The processor 114 may also include graphical processing units such as a GPU from the GeForce®, Quadro®, Tesla® family manufactured by Nvidia™, GMA, Iris™ family manufactured by Intel™, or the Radeon™ family manufactured by AMD™. The processor 114 may also include accelerated processing units such as the Xeon Phi™ family manufactured by Intel™. The disclosed embodiments are not limited to any type of processor(s) otherwise configured to meet the computing demands of identifying, analyzing, maintaining, generating, and/or providing large amounts of data or manipulating such data to perform the methods disclosed herein. In addition, the term “processor” may include more than one processor (for example, a multi-core design or a plurality of processors each having a multi-core design). The processor 114 can execute sequences of computer program instructions, stored in memory device 116, to perform various operations, processes, methods that will be explained in greater detail below.

The memory device 116 can store medical images 146. In some embodiments, the medical images 146 may include one or more MRI images (e.g., 2D MRI, 3D MRI, 2D streaming MRI, four-dimensional (4D) MRI, 4D volumetric MRI, 4D cine MRI, etc.), functional MRI images (e.g., fMRI, DCE-MRI, diffusion MRI), CT images (e.g., 2D CT, cone beam CT, 3D CT, 4D CT), ultrasound images (e.g., 2D ultrasound, 3D ultrasound, 4D ultrasound), one or more projection images representing views of an anatomy depicted in the MRI, synthetic CT (pseudo-CT), and/or CT images at different angles of a gantry relative to a patient axis, PET images, X-ray images, fluoroscopic images, radiotherapy portal images, SPECT images, computer generated synthetic images (e.g., pseudo-CT images), aperture images, graphical aperture image representations of MLC leaf positions at different gantry angles, and the like. Further, the medical images 146 may also include medical image data, for instance, training images, ground truth images, contoured images, and dose images. In an embodiment, the medical images 146 may be received from the image acquisition device 132. Accordingly, image acquisition device 132 may include an MRI imaging device, a CT imaging device, a CBCT imaging device, a PET imaging device, an ultrasound imaging device, a fluoroscopic device, a SPECT imaging device, an integrated linac and MRI imaging device, an integrated linac and CT imaging device, an integrated linac and CBCT imaging device, or other medical imaging devices for obtaining the medical images of the patient. The medical images 146 may be received and stored in any type of data or any type of format that the image processing device 112 may use to perform operations consistent with the disclosed embodiments.

The memory device 116 may be a non-transitory computer-readable medium, such as a read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a CD-ROM, a DVD or other optical storage, a cassette tape, other magnetic storage device, or any other non-transitory medium that may be used to store information including image, data, or computer-executable instructions (e.g., stored in any format) capable of being accessed by the processor 114, or any other type of computer device. The computer program instructions can be accessed by the processor 114, read from the ROM, or any other suitable memory location, and loaded into the RAM for execution by the processor 114. For example, the memory device 116 may store one or more software applications. Software applications stored in the memory device 116 may include, for example, an operating system 143 for common computer systems as well as for software-controlled devices. Further, the memory device 116 may store an entire software application, or only a part of a software application, that is executable by the processor 114. For example, the memory device 116 may store one or more radiation therapy treatment plans 142.

The image processing device 112 can communicate with the network 120 via the communication interface 118, which can be communicatively coupled to the processor 114 and the memory device 116. The communication interface 118 may provide communication connections between the image processing device 112 and radiotherapy system 100 components (e.g., permitting the exchange of data with external devices). For instance, the communication interface 118 may, in some embodiments, have appropriate interfacing circuitry to connect to the user interface 136, which may be a hardware keyboard, a keypad, or a touch screen through which a user may input information into radiotherapy system 100.

Communication interface 118 may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as a WiFi adaptor), a telecommunication adaptor (e.g., 3G, 4G/LTE and the like), and the like. Communication interface 118 may include one or more digital and/or analog communication devices that permit image processing device 112 to communicate with other machines and devices, such as remotely located components, via the network 120.

The network 120 may provide the functionality of a local area network (LAN), a wireless network, a cloud computing environment (e.g., software as a service, platform as a service, infrastructure as a service, etc.), a client-server, a wide area network (WAN), and the like. For example, network 120 may be a LAN or a WAN that may include other systems S1 (138), S2 (140), and S3 (141). Systems S1, S2, and S3 may be identical to image processing device 112 or may be different systems. In some embodiments, one or more systems in network 120 may form a distributed computing/simulation environment that collaboratively performs the embodiments described herein. In some embodiments, one or more systems S1, S2, and S3 may include a CT scanner that obtains CT images (e.g., medical images 146). In addition, network 120 may be connected to Internet 122 to communicate with servers and clients that reside remotely on the interne.

Therefore, network 120 can allow data transmission between the image processing device 112 and a number of various other systems and devices, such as the OIS 128, the radiation therapy device 130, and the image acquisition device 132. Further, data generated by the OIS 128 and/or the image acquisition device 132 may be stored in the memory device 116, the database 124, and/or the hospital database 126. The data may be transmitted/received via network 120, through communication interface 118 in order to be accessed by the processor 114, as required.

The image processing device 112 may communicate with database 124 through network 120 to send/receive a plurality of various types of data stored on database 124. For example, database 124 may include machine data (control points) that includes information associated with a radiation therapy device 130, image acquisition device 132, or other machines relevant to radiotherapy. Machine data information may include control points, such as radiation beam size, arc placement, beam on and off time duration, machine parameters, segments, MLC configuration, gantry speed, MRI pulse sequence, and the like. Database 124 may be a storage device and may be equipped with appropriate database administration software programs. One skilled in the art would appreciate that database 124 may include a plurality of devices located either in a central or a distributed manner.

In some embodiments, database 124 may include a processor-readable storage medium (not shown). While the processor-readable storage medium in an embodiment may be a single medium, the term “processor-readable storage medium” should be taken to include a single medium or multiple media (e.g., a centralized or distributed database, and/or associated caches and servers) that store the one or more sets of computer-executable instructions or data. The term “processor-readable storage medium” shall also be taken to include any medium that is capable of storing or encoding a set of instructions for execution by a processor and that cause the processor to perform any one or more of the methodologies of the present disclosure. The term “processor-readable storage medium” shall accordingly be taken to include, but not be limited to, solid-state memories and optical and magnetic media. For example, the processor-readable storage medium can be one or more volatile, non-transitory, or non-volatile tangible computer-readable media.

Image processor 114 may communicate with database 124 to read images into memory device 116 or store images from memory device 116 to database 124. For example, the database 124 may be configured to store a plurality of images (e.g., 3D MRI, 4D MRI, 2D MRI slice images, CT images, 2D Fluoroscopy images, X-ray images, raw data from MR scans or CT scans, Digital Imaging and Communications in Medicine (DIMCOM) data, projection images, graphical aperture images, etc.) that the database 124 received from image acquisition device 132. Database 124 may store data to be used by the image processor 114 when executing software program 144 or when creating radiation therapy treatment plans 142. Database 124 may store the data produced by the trained machine learning mode, such as a neural network including the network parameters constituting the model learned by the network and the resulting estimated data. As referred to herein, “estimate” or “estimated” can be used interchangeably with “predict” or “predicted” and should be understood to have the same meaning. The image processing device 112 may receive the imaging data, such as a medical image 146 (e.g., 2D MRI slice images, CT images, 2D Fluoroscopy images, X-ray images, 3DMRI images, 4D MRI images, projection images, graphical aperture images, image contours, etc.) from the database 124, the radiation therapy device 130 (e.g., an MR-linac), and/or the image acquisition device 132 to generate a treatment plan 142.

In an embodiment, the radiotherapy system 100 can include an image acquisition device 132 that can acquire medical images (e.g., MRI images, 3D MRI, 2D streaming MRI, 4D volumetric MRI, CT images, cone-Beam CT, PET images, functional MRI images (e.g., fMRI, DCE-MRI and diffusion MRI), X-ray images, fluoroscopic image, ultrasound images, radiotherapy portal images, SPECT images, and the like) of the patient. Image acquisition device 132 may, for example, be an MRI imaging device, a CT imaging device, a PET imaging device, an ultrasound device, a fluoroscopic device, a SPECT imaging device, or any other suitable medical imaging device for obtaining one or more medical images of the patient. Images acquired by the image acquisition device 132 can be stored within database 124 as either imaging data and/or test data. By way of example, the images acquired by the image acquisition device 132 can be also stored by the image processing device 112 as medical images 146 in memory device 116.

In an embodiment, for example, the image acquisition device 132 may be integrated with the radiation therapy device 130 as a single apparatus (e.g., an MR-linac). Such an MR-linac can be used, for example, to determine a location of a target organ or a target tumor in the patient, so as to direct radiation therapy accurately according to the radiation therapy treatment plan 142 to a predetermined target.

The image acquisition device 132 can be configured to acquire one or more images of the patient's anatomy for a region of interest (e.g., a target organ, a target tumor, or both). Each image, typically a 2D image or slice, can include one or more parameters (e.g., a 2D slice thickness, an orientation, and a location, etc.). In an embodiment, the image acquisition device 132 can acquire a 2D slice in any orientation. For example, an orientation of the 2D slice can include a sagittal orientation, a coronal orientation, or an axial orientation. The processor 114 can adjust one or more parameters, such as the thickness and/or orientation of the 2D slice, to include the target organ and/or target tumor. In an embodiment, 2D slices can be determined from information such as a 3D MRI volume. Such 2D slices can be acquired by the image acquisition device 132 in “real-time” while a patient is undergoing radiation therapy treatment, for example, when using the radiation therapy device 130, with “real-time” meaning acquiring the data in at least milliseconds or less.

The image processing device 112 may generate and store radiation therapy treatment plans 142 for one or more patients. The radiation therapy treatment plans 142 may provide information about a particular radiation dose to be applied to each patient. The radiation therapy treatment plans 142 may also include other radiotherapy information, such as control points including beam angles, gantry angles, beam intensity, dose-histogram-volume information, the number of radiation beams to be used during therapy, the dose per beam, and the like.

The image processor 114 may generate the radiation therapy treatment plan 142 by using software programs 144 such as treatment planning software (such as Monaco®, manufactured by Elekta AB of Stockholm, Sweden). In order to generate the radiation therapy treatment plans 142, the image processor 114 may communicate with the image acquisition device 132 (e.g., a CT device, an MRI device, a PET device, an X-ray device, an ultrasound device, etc.) to access images of the patient and to delineate a target, such as a tumor, to generate contours of the images. In some embodiments, the delineation of one or more OARs, such as healthy tissue surrounding the tumor or in close proximity to the tumor, may be required. Therefore, segmentation of the OAR may be performed when the OAR is close to the target tumor. In addition, if the target tumor is close to the OAR (e.g., prostate in near proximity to the bladder and rectum), then by segmenting the OAR from the tumor, the radiotherapy system 100 may study the dose distribution not only in the target but also in the OAR.

In order to delineate a target organ or a target tumor from the OAR, medical images, such as MRI images, CT images, PET images, fMRI images, X-ray images, ultrasound images, radiotherapy portal images, SPECT images, and the like, of the patient undergoing radiotherapy may be obtained non-invasively by the image acquisition device 132 to reveal the internal structure of a body part. Based on the information from the medical images, a 3D structure of the relevant anatomical portion may be obtained and used to generate a contour of the image. Contours of the image can include data overlaid on top of the image that delineates one or more structures of the anatomy. In some cases, the contours can be files associated with respective images that specify the coordinates or 2D or 3D locations of various structures of the anatomy depicted in the images.

In addition, during a treatment planning process, many parameters may be taken into consideration to achieve a balance between efficient treatment of the target tumor (e.g., such that the target tumor receives enough radiation dose for an effective therapy) and low irradiation of the OAR(s) (e.g., the OAR(s) receives as low a radiation dose as possible). Other parameters that may be considered include the location of the target organ and the target tumor, the location of the OAR, and the movement of the target in relation to the OAR. For example, the 3D structure may be obtained by contouring the target or contouring the OAR within each 2D layer or slice of an MRI or CT image and combining the contour of each 2D layer or slice. The contour may be generated manually (e.g., by a physician, dosimetrist, or health care worker using a program such as MONACO™ manufactured by Elekta AB of Stockholm, Sweden) or automatically (e.g., using a program such as the Atlas-based auto-segmentation software, ABAS™, manufactured by Elekta AB of Stockholm, Sweden). In certain embodiments, the 3D structure of a target tumor or an OAR may be generated automatically by the treatment planning software.

After the target tumor and the OAR(s) have been located and delineated, a dosimetrist, physician, or healthcare worker may determine a dose of radiation to be applied to the target tumor, as well as any maximum amounts of dose that may be received by the OAR proximate to the tumor (e.g., left and right parotid, optic nerves, eyes, lens, inner ears, spinal cord, brain stem, and the like). After the radiation dose is determined for each anatomical structure (e.g., target tumor, OAR), a process known as inverse planning may be performed to determine one or more treatment plan parameters that would achieve the desired radiation dose distribution. Examples of treatment plan parameters include volume delineation parameters (e.g., which define target volumes, contour sensitive structures, etc.), margins around the target tumor and OARs, beam angle selection, collimator settings, and beam-on times. During the inverse-planning process, the physician may define dose constraint parameters that set bounds on how much radiation an OAR may receive (e.g., defining full dose to the tumor target and zero dose to any OAR; defining 95% of dose to the target tumor; defining that the spinal cord, brain stem, and optic structures receive ≤45 Gy, ≤55 Gy and <54 Gy, respectively). The result of inverse planning may constitute a radiation therapy treatment plan 142 that may be stored in memory device 116 or database 124. Some of these treatment parameters may be correlated. For example, tuning one parameter (e.g., weights for different objectives, such as increasing the dose to the target tumor) in an attempt to change the treatment plan may affect at least one other parameter, which in turn may result in the development of a different treatment plan. Thus, the image processing device 112 can generate a tailored radiation therapy treatment plan 142 having these parameters in order for the radiation therapy device 130 to provide radiotherapy treatment to the patient.

In addition, the radiotherapy system 100 may include a display device 134 and a user interface 136. The display device 134 may include one or more display screens that display medical images, interface information, treatment planning parameters (e.g., projection images, graphical aperture images, contours, dosages, beam angles, etc.) treatment plans, a target, localizing a target and/or tracking a target, or any related information to the user. The user interface 136 may be a keyboard, a keypad, a touch screen or any type of device that a user may use to input information to radiotherapy system 100. Alternatively, the display device 134 and the user interface 136 may be integrated into a device such as a tablet computer (e.g., Apple iPad®, Lenovo Thinkpad®, Samsung Galaxy®, etc.).

Furthermore, any and all components of the radiotherapy system 100 may be implemented as a virtual machine (e.g., VMWare, Hyper-V, and the like). For instance, a virtual machine can be software that functions as hardware. Therefore, a virtual machine can include at least one or more virtual processors, one or more virtual memories, and one or more virtual communication interfaces that together function as hardware. For example, the image processing device 112, the OIS 128, and the image acquisition device 132 could be implemented as a virtual machine. Given the processing power, memory, and computational capability available, the entire radiotherapy system 100 could be implemented as a virtual machine.

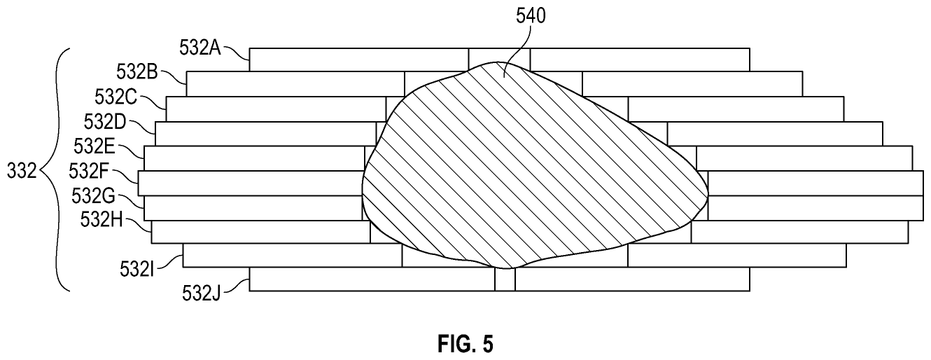

FIG. 2A illustrates an example of a radiation therapy device 202 that may include a radiation source, such as an X-ray source or a linear accelerator, a couch 216, an imaging detector 214, and a radiation therapy output 204. The radiation therapy device 202 may be configured to emit a radiation beam 208 to provide therapy to a patient. The radiation therapy output 204 can include one or more attenuators or collimators, such as an MLC as described in the illustrative embodiment of FIG. 5, below.

Referring back to FIG. 2A, a patient can be positioned in a region 212 and supported by the treatment couch 216 to receive a radiation therapy dose, according to a radiation therapy treatment plan. The radiation therapy output 204 can be mounted or attached to a gantry 206 or other mechanical support. One or more chassis motors (not shown) may rotate the gantry 206 and the radiation therapy output 204 around couch 216 when the couch 216 is inserted into the treatment area. In an embodiment, gantry 206 may be continuously rotatable around couch 216 when the couch 216 is inserted into the treatment area. In another embodiment, gantry 206 may rotate to a predetermined position when the couch 216 is inserted into the treatment area. For example, the gantry 206 can be configured to rotate the therapy output 204 around an axis (“A”). Both the couch 216 and the radiation therapy output 204 can be independently moveable to other positions around the patient, such as moveable in transverse direction (“T”), moveable in a lateral direction (“L”), or as rotation about one or more other axes, such as rotation about a transverse axis (indicated as “R”). A controller communicatively connected to one or more actuators (not shown) may control the couch's 216 movements or rotations in order to properly position the patient in or out of the radiation beam 208 according to a radiation therapy treatment plan. Both the couch 216 and the gantry 206 are independently moveable from one another in multiple degrees of freedom, which allows the patient to be positioned such that the radiation beam 208 can precisely target the tumor. The MLC may be integrated and included within gantry 206 to deliver the radiation beam 208 of a certain shape.

The coordinate system (including axes A, T, and L) shown in FIG. 2A can have an origin located at an isocenter 210. The isocenter 210 can be defined as a location where the central axis of the radiation beam 208 intersects the origin of a coordinate axis, such as to deliver a prescribed radiation dose to a location on or within a patient. Alternatively, the isocenter 210 can be defined as a location where the central axis of the radiation beam 208 intersects the patient for various rotational positions of the radiation therapy output 204 as positioned by the gantry 206 around the axis A. As discussed herein, the gantry angle corresponds to the position of gantry 206 relative to axis A, although any other axis or combination of axes can be referenced and used to determine the gantry angle.

Gantry 206 may also have an attached imaging detector 214. The imaging detector 214 is preferably located opposite to the radiation source, and in an embodiment, the imaging detector 214 can be located within a field of the therapy beam 208.

The imaging detector 214 can be mounted on the gantry 206 (preferably opposite the radiation therapy output 204), such as to maintain alignment with the therapy beam 208. The imaging detector 214 rotates about the rotational axis as the gantry 206 rotates. In an embodiment, the imaging detector 214 can be a flat panel detector (e.g., a direct detector or a scintillator detector). In this manner, the imaging detector 214 can be used to monitor the therapy beam 208 or the imaging detector 214 can be used for imaging the patient's anatomy, such as portal imaging (e.g., to provide real X-ray measurements). The control circuitry of radiation therapy device 202 may be integrated within system 100 or remote from it.

In an illustrative embodiment, one or more of the couch 216, the therapy output 204, or the gantry 206 can be automatically positioned, and the therapy output 204 can establish the therapy beam 208 according to a specified dose for a particular therapy delivery instance. A sequence of therapy deliveries can be specified according to a radiation therapy treatment plan, such as using one or more different orientations or locations of the gantry 206, couch 216, or therapy output 204. The therapy deliveries can occur sequentially, but can intersect in a desired therapy locus on or within the patient, such as at the isocenter 210. A prescribed cumulative dose of radiation therapy can thereby be delivered to the therapy locus while damage to tissue near the therapy locus can be reduced or avoided.

As indicated above, this disclosure describes techniques that use surface camera imaging data combined with other a priori information, e.g., intrabody imaging data, to describe how a patient is moving in 4D. Intrabody imaging data can include computed tomography (CT) imaging data and cone-beam CT (CBCT) imaging data, for example.

Surface camera imaging data can be acquired using one or more surface cameras 138A-138C. FIG. 2A depicts one non-limiting example in which one or more surface cameras 138A, 138B can be affixed to a ceiling 215 in the therapy treatment room and/or one or more surface cameras 138C can be affixed to a wall 217 in the therapy treatment room. One or more of the surface cameras 138A-138C can acquire surface camera imaging data in real time. The surface camera imaging data from one or more of the surface cameras 138A-138C can then be transmitted to an image processing device, such as to the image processing device 112 of FIG. 1, to generate a model, as described in more detail below.

FIG. 2B illustrates an example of a radiation therapy device 202 that may include a combined linac and an imaging system, such as can include a CT imaging system. The radiation therapy device 202 can include an MLC (not shown). The CT imaging system can include an imaging X-ray source 218, such as providing X-ray energy in a kiloelectron-Volt (keV) energy range which can be used for imaging the patient's anatomy, such as portal imaging (e.g., to provide real X-ray measurements). The imaging X-ray source 218 can provide a fan-shaped and/or a conical beam 208 directed to an imaging detector 222, such as a flat panel detector. The radiation therapy device 202 can be similar to the system described in relation to FIG. 2A, such as including a radiation therapy output 204, a gantry 206, a couch 216, and another imaging detector 214 (such as a flat panel detector). The X-ray source 218 can provide a comparatively-lower-energy X-ray diagnostic beam, for imaging.

In the illustrative embodiment of FIG. 2B, the radiation therapy output 204 and the X-ray source 218 can be mounted on the same rotating gantry 206, rotationally-separated from each other by 90 degrees. In another embodiment, two or more X-ray sources can be mounted along the circumference of the gantry 206, such as each having its own detector arrangement to provide multiple angles of diagnostic imaging concurrently. Similarly, multiple radiation therapy outputs 204 can be provided.

FIG. 2B depicts another non-limiting example in which one or more surface cameras 138D can acquire surface camera imaging data. In the example shown in FIG. 2B, a surface camera 138D can be affixed to a frontside of a radiation therapy device 202, such as to a frontside of a CT bore and another surface camera can be affixed to a backside of the radiation therapy device 202, such as to a backside of a CT bore. In this manner, the surface cameras can provide a continuous view of the patient. The surface camera imaging data from the surface cameras, such as the surface camera 138D and a backside surface camera can then be transmitted to an image processing device, such as to the image processing device 112 of FIG. 1, to generate a model, as described in more detail below.

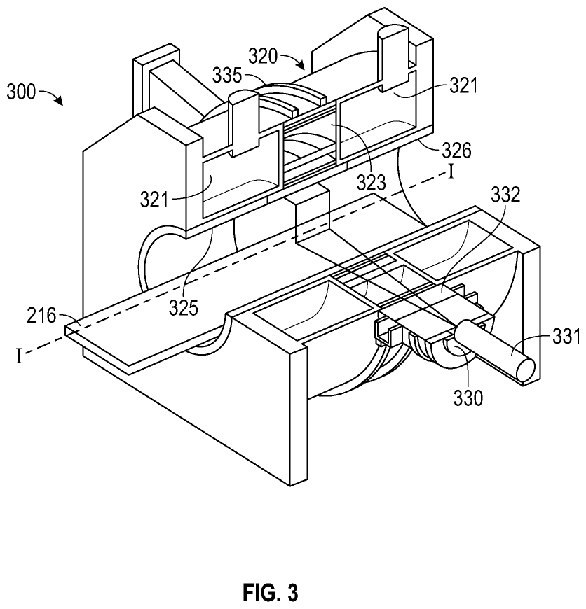

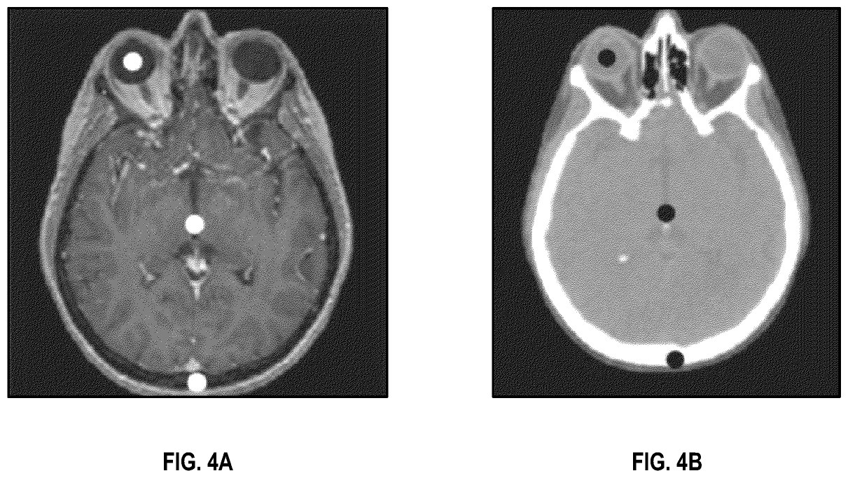

FIG. 3 depicts an example radiation therapy system 300 that can include combining a radiation therapy device 202 and an imaging system, such as a nuclear MR imaging system (e.g., known in the art as an MR-linac) consistent with the disclosed embodiments. As shown, system 300 may include a couch 216, an image acquisition device 320, and a radiation delivery device 330. System 300 delivers radiation therapy to a patient in accordance with a radiotherapy treatment plan. In some embodiments, image acquisition device 320 may correspond to image acquisition device 132 in FIG. 1 that may acquire origin images of a first modality (e.g., MRI image shown in FIG. 4A) or destination images of a second modality (e.g., CT image shown in FIG. 4B).

Couch 216 may support a patient (not shown) during a treatment session. In some implementations, couch 216 may move along a horizontal translation axis (labelled “I”), such that couch 216 can move the patient resting on couch 216 into and/or out of system 300. Couch 216 may also rotate around a central vertical axis of rotation, transverse to the translation axis. To allow such movement or rotation, couch 216 may have motors (not shown) enabling the couch 216 to move in various directions and to rotate along various axes. A controller (not shown) may control these movements or rotations in order to properly position the patient according to a treatment plan.

In some embodiments, image acquisition device 320 may include an MRI machine used to acquire 2D or 3D MRI images of the patient before, during, and/or after a treatment session. Image acquisition device 320 may include a magnet 321 for generating a primary magnetic field for magnetic resonance imaging. The magnetic field lines generated by operation of magnet 321 may run substantially parallel to the central translation axis I. Magnet 321 may include one or more coils with an axis that runs parallel to the translation axis I. In some embodiments, the one or more coils in magnet 321 may be spaced such that a central window 323 of magnet 321 is free of coils. In other embodiments, the coils in magnet 321 may be thin enough or of a reduced density such that they are substantially transparent to radiation of the wavelength generated by radiotherapy device 330. Image acquisition device 320 may also include one or more shielding coils, which may generate a magnetic field outside magnet 321 of approximately equal magnitude and opposite polarity in order to cancel or reduce any magnetic field outside of magnet 321. As described below, radiation source 331 of radiotherapy device 330 may be positioned in the region where the magnetic field is cancelled, at least to a first order, or reduced.

Image acquisition device 320 may also include two gradient coils 325 and 326, which may generate a gradient magnetic field that is superposed on the primary magnetic field. Coils 325 and 326 may generate a gradient in the resultant magnetic field that allows spatial encoding of the protons so that their position can be determined. Gradient coils 325 and 326 may be positioned around a common central axis with the magnet 321 and may be displaced along that central axis. The displacement may create a gap, or window, between coils 325 and 326. In embodiments where magnet 321 can also include a central window 323 between coils, the two windows may be aligned with each other.

In some embodiments, image acquisition device 320 may be an imaging device other than an MRI, such as an X-ray, a CT, a CBCT, a spiral CT, a PET, a SPECT, an optical tomography, a fluorescence imaging, ultrasound imaging, radiotherapy portal imaging device, or the like. As would be recognized by one of ordinary skill in the art, the above description of image acquisition device 320 concerns certain embodiments and is not intended to be limiting.

Radiotherapy device 330 may include the radiation source 331, such as an X-ray source or a linac, and an MLC 332 (shown below in FIG. 5). Radiotherapy device 330 may be mounted on a chassis 335. One or more chassis motors (not shown) may rotate chassis 335 around couch 216 when couch 216 is inserted into the treatment area. In an embodiment, chassis 335 may be continuously rotatable around couch 216 when couch 216 is inserted into the treatment area. Chassis 335 may also have an attached radiation detector (not shown), preferably located opposite to radiation source 331 and with the rotational axis of chassis 335 positioned between radiation source 331 and the detector. Further, device 330 may include control circuitry (not shown) used to control, for example, one or more of couch 216, image acquisition device 320, and radiotherapy device 330. The control circuitry of radiotherapy device 330 may be integrated within system 300 or remote from it.

During a radiotherapy treatment session, a patient may be positioned on couch 216. System 300 may then move couch 216 into the treatment area defined by magnet 321, coils 325 and 326, and chassis 335. Control circuitry may then control radiation source 331, MLC 332, and the chassis motor(s) to deliver radiation to the patient through the window between coils 325 and 326 according to a radiotherapy treatment plan.

FIG. 2A, FIG. 2B, and FIG. 3 illustrate generally embodiments of a radiation therapy device configured to provide radiotherapy treatment to a patient, including a configuration where a radiation therapy output can be rotated around a central axis (e.g., an axis “A”). Other radiation therapy output configurations can be used. For example, a radiation therapy output can be mounted to a robotic arm or manipulator having multiple degrees of freedom. In yet another embodiment, the therapy output can be fixed, such as located in a region laterally separated from the patient, and a platform supporting the patient can be used to align a radiation therapy isocenter with a specified target locus within the patient.

As discussed above, radiation therapy devices described by FIG. 2A, FIG. 2B, and FIG. 3 include an MLC for shaping, directing, or modulating an intensity of a radiation therapy beam to the specified target locus within the patient.

FIG. 5 illustrates an example of a MLC 332 that includes leaves 532A through 532J that can be automatically positioned to define an aperture approximating a tumor 540 cross section or projection. The leaves 532A through 532J permit modulation of the radiation therapy beam. The leaves 532A through 532J can be made of a material specified to attenuate or block the radiation beam in regions other than the aperture, in accordance with the radiation treatment plan. For example, the leaves 532A through 532J can include metallic plates, such as comprising tungsten, with a long axis of the plates oriented parallel to a beam direction and having ends oriented orthogonally to the beam direction (as shown in the plane of the illustration of FIG. 2A). A “state” of the MLC 332 can be adjusted adaptively during a course of radiation therapy treatment, such as to establish a therapy beam that better approximates a shape or location of the tumor 540 or another target locus. This is in comparison to using a static collimator configuration or as compared to using an MLC 332 configuration determined exclusively using an “offline” therapy planning technique. A radiation therapy technique using the MLC 332 to produce a specified radiation dose distribution to a tumor or to specific areas within a tumor can be referred to as IMRT.

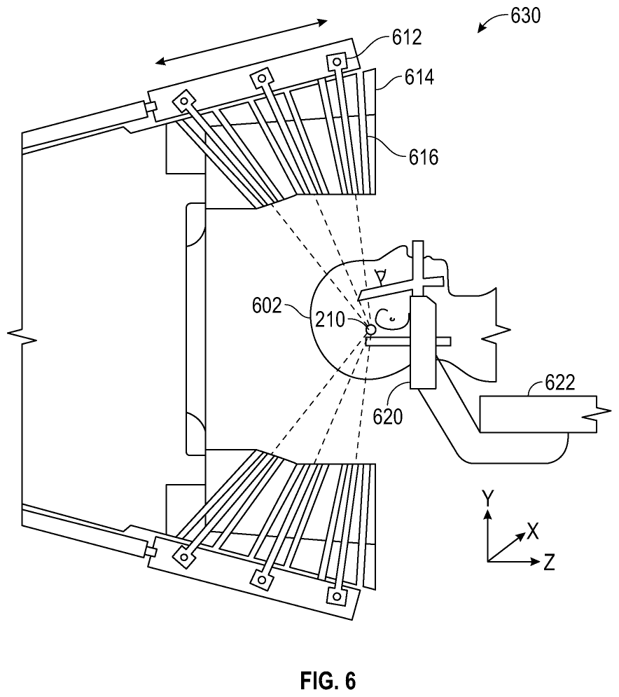

FIG. 6 illustrates an embodiment of another type of radiotherapy device 630 (e.g., a Leksell Gamma Knife), according to some embodiments of the present disclosure. As shown in FIG. 6, in a radiotherapy treatment session, a patient 602 may wear a coordinate frame 620 to keep stable the patient's body part (e.g., the head) undergoing surgery or radiotherapy. Coordinate frame 620 and a patient positioning system 622 may establish a spatial coordinate system, which may be used while imaging a patient or during radiation surgery.

Radiotherapy device 630 may include a protective housing 614 to enclose a plurality of radiation sources 612. Radiation sources 612 may generate a plurality of radiation beams (e.g., beamlets) through beam channels 616. The plurality of radiation beams may be configured to focus on an isocenter 210 from different directions. While each individual radiation beam may have a relatively low intensity, isocenter 210 may receive a relatively high level of radiation when multiple doses from different radiation beams accumulate at isocenter 210. In certain embodiments, isocenter 210 may correspond to a target under surgery or treatment, such as a tumor.

After initial images of the target are acquired, the location and/or orientation of the target region may change. For example, the patient may shift during transfer to the treatment room, during movement within the treatment room (e.g., positioning on a couch, bed, or table), or during the administration of radiotherapy. For example, a patient may have voluntarily or involuntarily movements due to regular biological processes, including, e.g., breathing, swallowing, blinking, twitching, peristalsis, digestion, beating of the heart, coughing, passing gas, or other movements.

Additionally, tracking anatomy across different radiation therapy treatment sessions (also referred to as fractions) can be complicated, because a patient can lose or gain weight between each fraction, a target region (e.g., tumor) can change size (e.g., shrink or get larger), or the anatomy around the target region can affect the position of the target region (e.g., the volume of a patient's bladder can change across fractions, affecting the location of surrounding structures).

These fraction-to-fraction changes in the location and/or orientation of the target region may reduce the efficacy of radiotherapy. For example, if the actual orientation or location of the target region is different than the assumed orientation or location based on prior imaging and/or inaccurate alignment with prior imaging, then the correct dose of radiation may not be delivered to the intended target region. Additionally, surrounding healthy structures can receive radiation instead of, or in addition to, the intended target region. Exposing the wrong area to radiation can ultimately harm or kill surrounding healthy cells. Further, it can be desirable to match images of the patient's anatomical structures taken across fractions and/or to an original image taken of the patient to track the location of dose delivery and the overall dose delivered to the patient.

Using various techniques of this disclosure, a system, such as the radiotherapy system 100 of FIG. 1, can acquire intrabody imaging information and surface information of a patient during a pre-treatment session. The surface information can either be acquired using a surface camera or extracted from the intrabody imaging information itself. The system can then generate a model that relates a 3D patient representation to 2D surface information. During a patient's treatment session, the system can acquire, e.g., in real time, surface information with one or more surface cameras and use the model to calculate the 3D patient representation in real time during the patient's treatment. In this manner, the system can drive the model using surface camera imaging data combined with other a priori information to describe how a patient is moving in 4D. The system can then modify a treatment plan dynamically using the 3D patient representation as input to account for any changes in the location and/or orientation of the target region.

In a first example, a radiation therapy system, such as the radiation therapy system 100 of FIG. 1, can be equipped with an integrated linac and CT imager, e.g., image acquisition device 132 of FIG. 1, and one or more surface cameras, e.g., one or more of surface cameras 138 of FIG. 1. The radiation therapy system can obtain intrabody imaging data and surface imaging data of the patient for determining a radiation treatment plan including a plurality of radiation treatment fraction sessions. For example, during CT image acquisition, the couch can be translated to acquire or otherwise obtain intrabody imaging data of a 3D representation of the patient (e.g., helical scan acquisition). As such, in some examples, the CT information need not be acquired during the treatment fraction session and instead can be acquired before initiating delivery of radiation therapy during that treatment fraction session.

As the couch is translated, e.g., rotated or otherwise moved, the CT imager can obtain, e.g., acquire, projections of the patient. The time of a respiratory cycle—from inhale to exhale—can be divided into a plurality of bins, e.g., 10 bins. The radiation therapy system can bin the obtained projections according to their position in the respiratory cycle. Once the radiation therapy system has binned enough projections to build a 3D image per bin, the radiation therapy system can generate 4D CT images of the patient.

In some examples, the surface camera(s) can be used to determine the bin of each projection and the radiation therapy system can determine where the projection is in the respiratory cycle and bin accordingly. In some examples, a surface camera can measure the positions of one or more reflective markers on the patient to determine phases of the respiratory cycle. In other examples, the CT images themselves can be used to determine where the projection is in the respiratory cycle.

By way of example, the patient can first be set up on the treatment couch, such as the couch 216 in FIG. 2B. A surface camera, such as the surface camera 138D in FIG. 2B, can be used to assist with this initial patient alignment. The radiation therapy system can then acquire a 4D CT image that provides a 3D CT image as a function of respiratory phase. The radiation therapy system can identify a reference phase, such as the one that has been used for the original treatment plan. For example, if maximum exhale was used during the original treatment plan, then maximum exhale can be used for the reference phase.

Surface imaging data, e.g., data representing the external surface of the patient during the reference phase, can be generated by extracting the surface imaging data from the CT image of the reference phase, where the CT image is a 3D image. Any technique that can extract a surface contour from a CT image can be used (e.g., as included in Elekta Monaco), such as threshold, active contour, atlas, or snake-based methods. In this manner, the radiation therapy system can determine a reference external surface representation corresponding to a reference respiratory phase bin.

Alternatively, surface imaging data can be found by deforming the external surface used for radiation treatment planning. For example, external contours of the patient can be found during treatment planning, such as by determining an external surface of the patient using an acquired diagnostic CT image. In some examples, the external surface can be binned according to respiration, such as by using a 4D CT imager for the treatment planning session. During the treatment planning session, the radiation therapy system can acquire a new CT image and determine a deformation vector field (DVF) between the initial CT image and the new CT image. The DVF can be used to warp the external contour to the new image.

In some examples, DVFs can be determined using grayscale information, such as without using any features. In some examples, the deformation can be a pixel-to-pixel (or voxel-to-voxel) deformation in time where each pixel (or voxel) can have a deformation vector that defines its movement from one 3D image to the next 3D image. If there is no deformation, all pixel (or voxel) deformation vectors are null. If there is deformation, the pixel (or voxel) deformation vectors point in various directions.

Next, the radiation therapy system can generate a model from the intrabody imaging data, e.g., 3D CT imaging data, and the surface imaging data to relate a 3D patient representation to a 2D surface patient representation. For example, the radiation therapy system can calculate DVFs between the intrabody imaging data, e.g., 3D CT imaging data, for each respiratory phase bin and the 3D CT imaging data of the reference phase bin using a deformable registration technique. The radiation therapy system can acquire 4D CT images of the day of treatment of the patient, e.g., 10 CT images with one CT image per respiratory phase bin. One of the CT images can be used as the reference image and the radiation therapy system can determine how the reference image deforms to each of the CT images in the other respiratory phase bins. In this manner, the radiation therapy system can calculate DVFs, e.g., 10 DVFs corresponding to the 10 respiratory phase bins, to find the deformation between each successive 3D image data volume and the reference 3D image data volume. Thus, the radiation therapy system can determine a first DVF between various respiratory phase bins and the reference respiratory phase bin.

The radiation therapy system can extract the points of the first DVF that correspond to the patient surface. Continuing the non-limiting example above, there can be 10 DVFs corresponding to the 10 respiratory phase bins. The radiation therapy system can determine from the 10 DVFs an external surface DVF that includes external surface points only and an internal DVF that includes the patient anatomy. The radiation therapy system can determine a second DVF corresponding to an external surface associated with the first DVF. Thus, there are two DVFs—the patient anatomy “DVFint,” and the patient external “DVFext” for only the points on the external surface, where DVFext is a subset of DVFint such that DVFint includes the vectors of DVFext.

After the radiation therapy system calculates the two DVFs, e.g., DVFint and DVFext, the radiation therapy system has a set of DVFs that describe how the organ moves, e.g., translates and/or rotates, and/or deforms during respiration. The set of DVFs can include a substantial amount of information, which can be computationally difficult to process. To simply the computation, a processor, such as the image processor 114 of FIG. 1, can reduce the dimensionality of the set of DVFs, if desired.

For example, the image processor 114 of FIG. 1 can apply a dimensionality reduction technique to the DVFs. The dimensionality reduction technique can include applying a principal component analysis (PCA) to the DVFs. Application of PCA to the DVFs results in a set of principal components or coefficients, which define vectors. Then, using a predefined criterion, such as a predefined amount of variability, or a predefined desired accuracy of a reconstructed deformation field, the radiation therapy system can reduce the dimensionality by selecting one or more PCA components from the set of principal components. In an example, the accuracy can be defined as a measure of the difference between a reconstructed deformation field and the ones given by the registration.

Dimensionality reduction techniques are not limited to the use of PCA. Other non-limiting examples of dimensionality reduction techniques include independent component analysis (ICA), kernel PCA, canonical correlation analysis, locally linear embedding (LLE), Hessian LLE, Laplacian eigenmaps, local tangent space alignment, maximum variance unfolding, and maximally informative dimensions.

In some examples, the radiation therapy system can perform principal component analysis (PCA) to generate a model using at least one of the first DVF or the second DVF. For example, a processor of the radiation therapy system can perform principal component analysis of both DVFs, e.g., DVFint and DVFext, and the first few components (e.g., 2 or 3) can be kept because that is generally sufficient for respiratory motion; the other components are primarily noise.

For one or more of the respiratory bins, the radiation therapy system can determine an internal PCA, “PCA-DVFint” and an external PCA, “PCA-DVFext” that correspond to DVFint and DVFext, respectively. The processor can determine a model that relates the PCA components of PCA-DVFext and PCA-DVFint—for example, a linear model, such as described in US20170360325, or a machine learning regression model such as described in US2020129780. The model allows the radiation therapy system to take the original reference phase and deform it by a DVF to determine what the CT image looks like in real-time at a particular instant in time.

During treatment, at each time point, the patient's surface can be acquired with the surface camera(s). The DVF “DVFext” between the surface and the reference surface can be determined by the radiation therapy system, and the radiation therapy system can determine the PCA components of this DVF as “PCA-DVFext”. The previously generated model can then be used to calculate the PCA components “PCA-DVFint” from DVF “DVFext”, where this “DVFext” is “of the moment” during the treatment in contrast to the “DVFext” above that was used to generate the model. For example, the previously generated model can be used to determine the DVF “DVFint” that is “of the moment” from the DVF “DVFext” that is “of the moment” and then the PCA components “PCA-DVFint” can be determined from the DVF “DVFint”. In this manner, the radiation therapy system can determine, using the model, at least one DVF during the particular radiation treatment fraction session.

Once the PCA components are determined (“PCA-DVFint”) from the DVF “DVFext”, then the radiation therapy system can relate the PCA-DVFint to the model using the PCA components of the DVF “DVFint”, which allows the radiation therapy system to calculate the DVF “DVFint” itself. That is, the radiation therapy system can use the model to generate the first DVF from the second DVF to generate an intrafractional intrabody image representation of the patient at various times during a particular radiation treatment fraction session.

Then, the radiation therapy system can deform the original reference phase of the 4D CT image by the DVF “DVFint” to determine what the CT image looks like in real-time. This deformed CT image can be used as the ‘current’ instantaneous CT image representation of the patient. In this way, a full real-time CT image can be determined. The radiation therapy system can use the generated intrafractional intrabody image representation of the patient to modify one or more parameters, e.g., treatment parameters, of the radiation therapy delivered during that particular radiation treatment fraction session.

A structure set is a set of all the contours of relevant anatomy including target and organs-at-risk, such as prostate, bladder, and rectum. They are important for treatment planning, for example to calculate dose-volume histograms for each structure, to guide plan optimization algorithms, etc.

Since the DVFs are determined in real-time in this approach, they can also be used to deform the structure set of the session to the current time point. Both the instantaneous CT and structure set can then be used as inputs, such as to dynamically adjust the treatment plan.

In another example, rather than equipping the linear accelerator with an integrated CT imager and a surface camera, a radiation therapy system, such as the radiation therapy system 100 of FIG. 1, can be equipped with a CBCT imager, e.g., image acquisition device 132 of FIG. 1, and one or more surface cameras, e.g., one or more of surface cameras 138 of FIG. 1. A similar technique as described above can be used, such as with the following modifications:
a) A 4D CBCT image can be acquired instead of a 4D CT image. The CBCT images can be acquired during treatment because the couch does not need to be moved.
b) The 4D CBCT image can optionally be used to calculate a synthetic 4D CT image, such as by applying a technique to calculate synthetic CT images (sCTs) from CBCTs. A synthetic CT image technique can be used to transform a first kind of image of to a second kind of image, such as by using DVFs or artificial intelligence techniques. Each phase of a 4D CBCT image can be transformed into a sCT image.
c) If the 4D CBCT image has limited FOV, surface camera information can be acquired concurrently with 4D CBCT and used to generate an expanded 4D CBCT or a synthetic 4D CT to fill in any gaps due to the limited FOV, such as described in U.S. Provisional Application No. 63/066,542 (Attorney Docket No. 4186.151PRV; Client Ref. No. 20US02, entitled “Patient imaging for online adaptive radiotherapy”, which is incorporated by reference herein), and described in detail below.
d) Either the instantaneous CBCT image can be calculated (such as described using the techniques above when an instantaneous CT image was used), and then used to calculate the instantaneous synthetic CT if desired, or if a synthetic CTs had been used to generate the model, the instantaneous synthetic CT can be evaluated directly if the model instead of CBCTs.

In another embodiment, rather than use a model to relate DVFext and DVFint (or PCA-DVFext and PCA-DVFint), a direct analytical expression can be used because the DVF “DVFext” is a subset of the vectors in the DVF “DVFint” (as opposed to when kV images or MR slices are used, where no such relation exists). For example, a set of linear equations can be written, such as can relate a finite number of “DVFext” points to the PCA components of the DVF “DVFint”. This set of linear equations can be solved exactly when the number of linear equations is sufficient for the number of PCA components being solved for. If there are more linear equations than necessary, than an optimization technique can be used to find the best-fit solution. In some other approaches, the PCA-DVFint components that lead to surface points that have a best match to the external surface contour can be calculated.

In some embodiments, the deep learning techniques described in US20200160972 and U.S. Provisional Application No. 62/991,356, (Attorney Docket No. 4186.139PRV; Client Ref. No. 19US11PROV), filed Mar. 18, 2020, can be applied instead of the DVF method, replacing kV data with surface data, with each of these patent documents being incorporated by reference herein. For example, a machine learning model can be trained with patient image and patient external surface pairs such that the machine learning model can generate an image based on a particular external surface.

In some embodiments, rather than generate a 4D CT image or 4D CBCT image, x-ray projection data can be acquired concurrently with surface camera data before delivering radiation treatment. This can be used in building a deep learning model directly without the interim step of generating a 4D image, calculating DVFs, and PCAs of the DVFs.

One concern with this surface-guided approach is that the model of surface and internal anatomy can degrade with time during a treatment session. Correlation models between a few points on the patient's surface and a single target centroid point can deteriorate with time. The approach can utilize multiple surface points and a full image, such that there is overlap between the surface points and the full image (the points on the surface are also part of the image). Thus, using the present approach, the model can remain valid for longer periods of time. If high radiation dose rates are used, limiting treatment times, then one can use the model with relatively high confidence. If treatment times are not short, then the treatment can be subdivided into many shorter sub-fractions, with a new 4D CBCT image being acquired, and a new model being re-generated for each sub-fraction.

Alternatively, concurrently with surface data acquisition, another modality such as kV or a single CT slice can be used to validate the model, and the model can be re-trained if the validation fails.

This disclosure has focused on enabling dynamic adaptive radiotherapy, such as during a treatment fraction session. In some cases, however, the images generated need not be used to adapt the treatment plan, but can be used for retrospective dose calculations, for gating a radiation treatment beam based on excursions of the target or organ-at-risk (OAR) from their reference positions, or for geometrically modifying leaf positions of a multi-leaf collimator (MLC) used for directing the radiation treatment beam, such as based on target or OAR excursions.

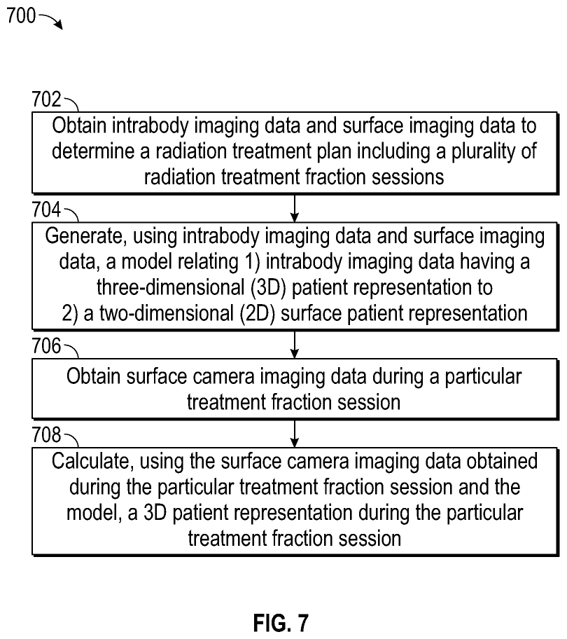

FIG. 7 illustrates a flow diagram of an example of a computer-assisted radiation treatment planning and treatment delivery method 700, according to some embodiments of the present disclosure. At block 702, a radiation therapy system, such as the radiation therapy system 100 shown in FIG. 1, can acquire or otherwise obtain intrabody imaging data and surface imaging data for determining a radiation treatment plan including a plurality of radiation treatment fraction sessions.

For example, the radiation therapy system can obtain intrabody imaging data using an image acquisition device, such as the image acquisition device 132 of FIG. 1. The image acquisition device can acquire intrabody imaging data such as computed tomography (CT) imaging data and cone-beam CT (CBCT) imaging data, for example, such as during a treatment fraction session prior to delivery of radiation therapy. In some examples, the radiation therapy system can obtain the intrabody imaging data during a treatment fraction session before initiating delivery of radiation therapy during that treatment fraction session.

The radiation therapy system can acquire or otherwise obtain surface imaging data of the patient, e.g., data representing the external surface of the patient during a reference phase, such as by extracting the surface imaging data from the intrabody imaging data, e.g., CT imaging data or CBCT imaging data, of the reference phase, where the intrabody image is a 3D image. Alternatively, surface imaging data can be found by deforming the external surface used for radiation treatment planning. For example, external contours of the patient can be found during treatment planning, such as by determining an external surface of the patient using an acquired diagnostic CT image. In other examples, one or more surface cameras, such as one or more surface cameras 138 of FIG. 1, can be used to generate the surface imaging data.

At block 704, the radiation therapy system, e.g., the processor 112 of the radiation therapy system 100 of FIG. 1, can use the intrabody imaging data and the surface imaging data to generate a model relating 1) the intrabody imaging data, e.g., from a CT image or CBCT image, having a three-dimensional (3D) patient representation to 2) a two-dimensional (2D) surface patient representation, e.g., from the surface imaging data of the patient. For example, as described above, the radiation therapy system can calculate DVFs between the intrabody imaging data, e.g., 3D CT imaging data, for each respiratory phase bin and the 3D CT imaging data of the reference phase bin using a deformable registration technique, perform a PCA of the internal and external surface DVFs, and generate a model that relates the PCA components.

At block 706, the radiation therapy system can obtain surface camera imaging data during a particular treatment fraction session, such as by using one or more surface cameras 138 of FIG. 1. For example, the radiation therapy device 202 of FIG. 2 can use one or more of the surface cameras 138A-138C to obtain surface camera imaging data during a particular treatment fraction session. During treatment, at various time points, the patient surface can be acquired with the surface camera.

At block 708, the radiation therapy system can use the surface camera imaging data obtained during the particular treatment fraction session and the model to calculate a 3D patient representation during the particular treatment fraction session. For example, the DVF “DVFext” between the surface and the reference surface can be determined by the radiation therapy system, and the radiation therapy system can determine the PCA components of this DVF as “PCA-DVFext”. The previously generated model (from block 704) can then be used to calculate the PCA components “PCA-DVFint” from DVF “DVFext”, where this “DVFext” is “of the moment” during the treatment in contrast to the “DVFext” above that was used to generate the model.

Once the PCA components are determined (“PCA-DVFint”) from the DVF “DVFext”, then the radiation therapy system can relate the PCA-DVFint to the model using the PCA components of the DVF “DVFint”, which allows the radiation therapy system to calculate the DVF “DVFint” itself. Then, the radiation therapy system can deform the original reference phase of the 4D CT image by the DVF “DVFint” to determine what the CT image looks like in real-time. This deformed CT can be used as the ‘current’ instantaneous CT representation of the patient.

In this way, a full real-time CT image can be determined to assist in determining a radiation treatment plan. In some examples, the method 700 of FIG. 7 can include modifying one or more parameters, e.g., radiation treatment parameters, of the radiation treatment plan for the particular treatment fraction session during that particular treatment fraction session. For example, the radiation therapy system can modify one or more parameters, e.g., radiation treatment parameters, of to account for intrafraction motion by adjusting the couch, adjusting the dose rate, adjusting the gantry, adjusting one or more leaves of the MLC, adjusting one or more parameters of the linac, and the like during the session itself in order to provide accurate, real-time, 3D localization and tracking of a target, e.g., tumor.

In some examples, the method 700 can include binning projections from intrabody imaging data to create a 4D image. For example, as the couch is translated, e.g., rotated or otherwise moved, the image acquisition device, such as the image acquisition device 132 of FIG. 1 can obtain projections of the patient. The radiation therapy system can bin the obtained projections according to the respiratory cycle. In some examples, the 4D image can provide a 3D image over various respiratory phases. That is, once the radiation therapy system has binned enough projections to build a 3D image per bin, the radiation therapy system can generate 4D CT images of the patient.

In some examples, the radiation therapy system can determine a reference external surface representation corresponding to a reference respiratory phase bin, determine a first deformation vector field (DVF) between various respiratory phase bins and the reference respiratory phase bin, and determine a second DVF corresponding to an external surface associated with the first DVF.

In some examples, the radiation therapy system can use the model to generate the first DVF from the second DVF for generating an intrafractional intrabody image representation of the patient at various times during a particular radiation treatment fraction session. In an example, the radiation therapy system can use the generated intrafractional intrabody image representation of the patient to modify radiation therapy then delivered during that particular radiation treatment fraction session.

As mentioned above, rather than using a 4D CT image initially, the radiation therapy system acquiring a 4D CBCT image instead of a 4D CT image in the method 700. The 4D CBCT images can be acquired during treatment because the couch does not need to be moved.

Then, the radiation therapy system can generate a synthetic 4D CT image representation from the 4D CBCT image. The radiation therapy system can apply a synthetic CT (sCT) image technique to transform a CBCT image to a CT image, such as by using DVFs or artificial intelligence techniques. Each phase of a 4D CBCT image can be transformed into a sCT image.

If the 4D CBCT image has limited FOV, surface camera information can be acquired concurrently with 4D CBCT to augment the 4D CBCT image with surface camera imaging data, as described in detail below. Then, either the instantaneous CBCT image can be calculated (such as described using the techniques above when an instantaneous CT image was used), and then used to calculate the instantaneous synthetic CT if desired, or if any synthetic CTs had been used to generate the model, the instantaneous synthetic CT can be evaluated directly if the model instead of CBCTs. In this manner, the radiation therapy system can generate an intrafractional intrabody image representation of the patient at various times during a particular radiation treatment fraction session using the synthetic 4D CT image representation.

In some examples, the radiation therapy system can use the generated intrafractional intrabody image representation of the patient to modify one or more parameters, e.g., treatment parameters, of radiation therapy then delivered during that particular radiation treatment fraction session. For example, the radiation therapy system can modify one or more parameters by adjusting the couch, adjusting a timing of the delivery of the radiation therapy based on respiration phase, adjusting the gantry, adjusting one or more leaves of the MLC, adjusting parameters of the linac, and the like.

In addition to lower image quality, CBCT scanners can sometimes suffer from limited FOV, allowing only a central portion of the patient to be imaged. This may be sufficient for patient alignment purposes but does not provide the full patient anatomy required for treatment planning, limiting its use in adaptive radiotherapy. For adaptive radiotherapy, it can be desirable to have a complete representation of the patient including body contours. Thus, a sCT generated from a 4D CBCT with limited FOV can be of lower quality.

The present inventors have recognized a need to find a solution to sCT generation using limited FOV CBCT images. As described below, if the 4D CBCT image has limited FOV, surface camera information can be acquired concurrently with 4D CBCT and used to generate an expanded 4D CBCT or a synthetic 4D CT to fill in any gaps due to the limited FOV to account for changes within a fraction. Using these techniques, a surface camera combined with a limited FOV 4D CBCT image can be used to generate a complete representation of the patient for adaptive radiotherapy.

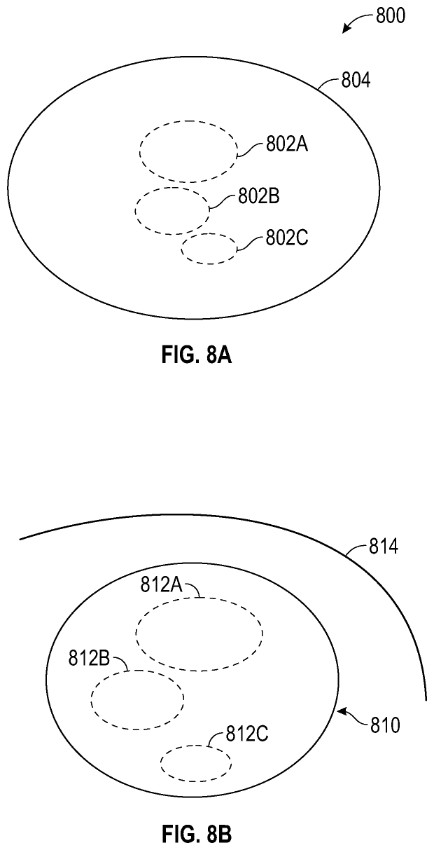

FIG. 8A depicts a conceptualized diagram of a planning CT image. The planning CT image 800 of a patient can include internal anatomy 802A-802C and an external CT contour 804. The planning CT image 800 can include pre-treatment CT imaging data acquired or otherwise obtained by a radiation therapy system, such as by the image acquisition device 132, e.g., CT imager, of the radiation therapy device 100 of FIG. 1.

FIG. 8B depicts a conceptualized diagram of a limited FOV CBCT acquired during a radiotherapy fraction as well as a partial external contour measured with the surface camera. The limited FOV CBCT image 810 depicts internal anatomy 812A-812C that corresponds to internal anatomy 802A-802C of FIG. 8B, but the internal anatomy has moved since the planning CT image was obtained. The limited FOV CBCT image 810 can include CBCT imaging data be acquired or otherwise obtained using a linear accelerator, such as can be equipped with an integrated CBCT imager, during a treatment fraction session.

The diagram in FIG. 8B further includes a partial external contour 814 defined using surface camera imaging data, such as acquired or otherwise obtained using a surface camera, such as using one or more surface cameras 138 of the radiation therapy device 100 of FIG. 1, such as during the treatment fraction session. The internal and external information are both in the linear accelerator's coordinate system.

The radiation therapy system can use the surface camera imaging data to reconstruct at least part of the patient's surface. It may not be possible to reconstruct the complete patient surface, such as due to line-of-sight issues that can be caused by patient positioning devices. Examples of surface cameras can include those manufactured by C-Rad, VisionRT, and Varian HumediQ. In another example, mm-wave technology can be used to reconstruct the full patient surface.

At this point, the radiation therapy system has obtained imaging data representing a full patient surface information (full body contour) at the time of treatment planning and imaging data representing a partial patient surface information (partial body contour) at the time of treatment. In some examples, it can be desirable to also obtain full patient surface information at the time of treatment.

In some examples, the radiation therapy system can obtain the full patient surface information at the time of treatment using millimeter-wave technology. In other examples, the radiation therapy system can obtain the full patient surface information at the time of treatment using multiple surface cameras concurrently, such as to provide more patient surface information than can be provided with just one surface camera. In yet other examples, the full patient surface information at the time of treatment can first be estimated by deforming the reference CT-based external contour so that it aligns with the surface camera contour, before calculating the DVF.

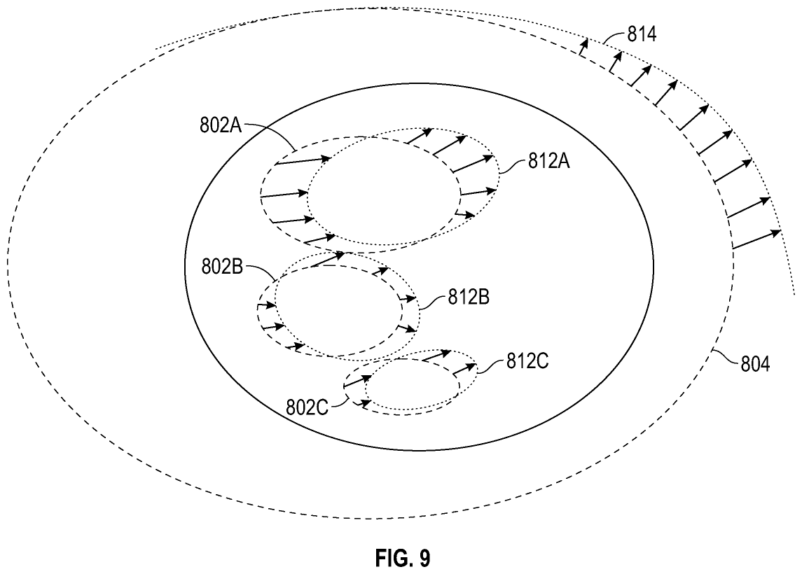

Next, the radiation therapy system can generate a synthetic CT (sCT), which synthetically attempts to replicate the acquisition of a new diagnostic CT. The radiation therapy system can generate sCT imaging data, such as by determining a deformation field vector (DVF) between the CT and CBCT imaging data, using one or more constraints or limitations from the surface camera imaging data. As described below, the radiation therapy system can determine DVFs that warp the surface points from the time of treatment planning, such as using the CT imaging data) to the surface points at the time of treatment, such as using the surface camera imaging data. A DVF can be determined such that the DVF ensures a) the voxels in the limited FOV CBCT are mapped to corresponding voxels in the CT, and b) points on the surface camera partial external contour are mapped to points on the external CT contour. Points that do not have some correspondence in both datasets may either remain stationary or can follow a smooth deformation that is consistent with the rest of the DVF. The radiation therapy system can then deform the CT image from the time of treatment planning using the DVF to produce the sCT image. A graphical example of warping using DVFs is shown in FIG. 9.

FIG. 9 is a conceptualized diagram illustrating how the surface points from the time of treatment planning in FIG. 8A relate to the surface points at the time of treatment in FIG. 8B. In FIG. 9, the various arrows represent the vectors of corresponding DVFs and depict the movement between a point on a surface from the time of treatment planning (FIG. 8A) and a corresponding point on a surface at the time of treatment (FIG. 8B). The arrows illustrate the movement of the internal anatomy 802A-802C in FIG. 8A as well as the movement of the external CT contour 804 in FIG. 8A from the time of treatment planning to the time of treatment.

In some examples, the radiation therapy system can determine DVFs that warp surface points from the planning contour of the diagnostic CT image taken at the time of treatment planning, such as using the CT imaging data, to corresponding surface points at the time of treatment, such as using the surface camera imaging data, to generate full patient surface information at the time of treatment.

There are many techniques that can be used to deform or warp one surface towards another. For example, the radiation therapy system can use a technique such as iterative closest point (ICP) that can continuously move/warp the points on the surfaces of the treatment planning image so that they match the points on the surfaces of the treatment image. In other examples, the radiation therapy system, e.g., the processor 114 of FIG. 1, can iteratively change the vectors of the DVF to deform one surface towards another. In yet other examples, the radiation therapy system, e.g., the processor 114 of FIG. 1, can deform one surface towards another by using a spline.

To recap, the radiation therapy system had a complete image, e.g., CT image, and full patient surface information at the time of treatment planning, such as obtained by using a CT imager, and a partial FOV image, e.g., CBCT image, and partial patient surface information, such as obtained using a CBCT image, at the time of treatment. Now, using the techniques described above, the radiation therapy system has a partial FOV image and full patient surface information at the time of treatment.

Once the radiation therapy system has deformed the surface 804 to match the surface 814 in FIG. 9, the radiation therapy system, e.g., the processor 114 of FIG. 1, can execute instructions to perform constrained optimization to determine a DVF that finds the best match between the deformed CBCT and CT voxels. For example, the processor can use the constraint or limitation that one or more points on the external contours must also match.

In some examples, the step of estimating the complete surface contour from incomplete surface data need not be performed. That is, the complete surface 804 in FIG. 9 is not needed. Rather, the radiation therapy system can perform constrained optimization to determine the DVF that finds the best match between the deformed CBCT and CT voxels, such as using the constraint or limitation that the partial surface camera information from the surface camera imaging data aligns with points on the external CT contour. That is, the radiation therapy system can constrain or limit the partial patient surface information so that the DVF is constrained or limited to the points that the radiation therapy system already has without having to determine the full patient surface information. The radiation therapy system can then deform the CT image from the time of treatment planning using the DVF to produce the sCT image.

In some examples, the radiation therapy system can bin projections according to the respiratory cycle, similar to the techniques described above. For example, for a patient being treated for a lung condition, it can be desirable to look at patient motion. The radiation therapy system can acquire a 4D CBCT image over a respiratory cycle. Once the radiation therapy system has binned enough projections to build a 3D image per bin, the radiation therapy system can select a particular bin corresponding to a respiratory phase of interest and use the corresponding 3D image as the image 810 in FIG. 8B. In other examples, the techniques can be used for more than one respiratory phase rather than with a selected respiratory phase. In this manner, the radiation therapy system can bin projections from the CBCT imaging data to create a 4D image. In some examples, the 4D image can provide a 3D image over various respiratory phases.

There are various other techniques that can be implemented as well, including deep learning techniques. For example, the radiation therapy system can perform the following: a) estimate the complete patient contour from partial surface camera data; b) fill in data pixels between the limited FOV CBCT and the estimated external surface with an average pixel value to generate an approximate ‘full FOV’ CBCT; and c) use a deep learning sCT algorithm, such as that implemented in ADMIRE, such as to find a sCT that corresponds to the approximate ‘full FOV’ CBCT.

Then, radiation therapy system can generate synthetic CT (sCT) imaging data from the CBCT imaging data and the surface camera imaging data.

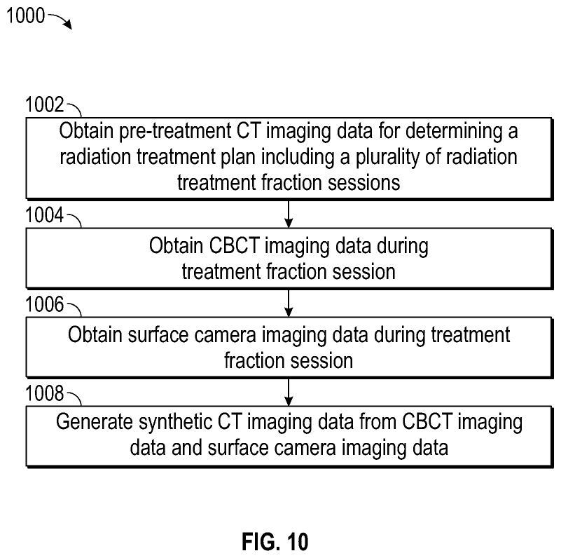

FIG. 10 illustrates a flow diagram of another example of a computer-assisted radiation treatment planning and treatment delivery method 1000, according to some embodiments of the present disclosure. At block 1002, a radiation therapy system, such as the radiation therapy system 100 shown in FIG. 1, can acquire or otherwise obtain pre-treatment CT imaging data for determining a radiation treatment plan including a plurality of radiation treatment fraction sessions.

For example, the radiation therapy system can obtain CT imaging data using an image acquisition device, such as the image acquisition device 132 of FIG. 1, during a treatment planning session. In some examples, the image acquisition device can acquire pre-treatment CT imaging data from a planning CT image. The planning CT image can include internal anatomy and an external CT contour, such as shown in FIG. 8A.

At block 1004, a radiation therapy system, such as the radiation therapy system 100 shown in FIG. 1, can acquire or otherwise obtain CBCT imaging data during a treatment fraction session. The CBCT imaging data can be acquired or otherwise obtained using a linear accelerator, such as can be equipped with an integrated CBCT imager. For example, the CBCT imaging data can be acquired or otherwise obtained by a radiation therapy system, such as by the image acquisition device 132, e.g., CBCT imager, of the radiation therapy device 100 of FIG. 1. The CBCT imaging data obtained at the time of treatment can represent a partial FOV image due to the limited FOV of the CBCT imager.

At block 1006, the radiation therapy system can obtain surface camera imaging data during the treatment fraction session, such as by using one or more surface cameras 138 of FIG. 1. For example, the radiation therapy device 202 of FIG. 2 can use one or more of the surface cameras 138A-138C to obtain surface camera imaging data during a particular treatment fraction session. During treatment, at each time point, the patient surface can be acquired with the surface camera(s).

At block 1008, the radiation therapy system can generate sCT imaging data from the CBCT imaging data and the surface camera imaging data. The sCT imaging data synthetically attempts to replicate the acquisition of a new diagnostic CT. The radiation therapy system can generate sCT imaging data, such as by determining a deformation field vector (DVF) between the CT and CBCT imaging data, using one or more constraints or limitations from the surface camera imaging data.

In some examples, the method 1000 can include modifying a radiation treatment plan using information derived from the DVF obtained during a particular treatment fraction session to modify a radiation treatment plan for that particular treatment fraction session or a later treatment fraction session.

In some examples, the method 1000 can include binning projections from the CBCT imaging data to create a 4D image. In some examples, the 4D image provides a 3D image over various respiratory phases.

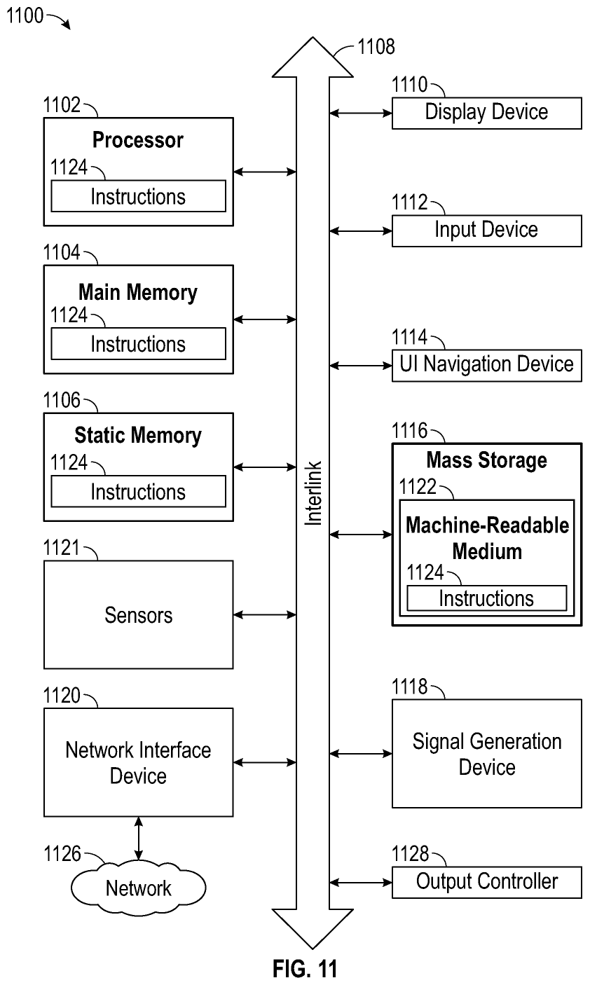

FIG. 11 illustrates a block diagram of an embodiment of a machine 1100 on which one or more of the methods as discussed herein can be implemented. In one or more embodiments, one or more items of the image processing device 112 can be implemented by the machine 1100. In alternative embodiments, the machine 1100 operates as a standalone device or may be connected (e.g., networked) to other machines. In one or more embodiments, the image processing device 112 can include one or more of the items of the machine 1100. In a networked deployment, the machine 1100 may operate in the capacity of a server or a client machine in server-client network environment, or as a peer machine in a peer-to-peer (or distributed) network environment. The machine 1100 may be a personal computer (PC), a tablet PC, a set-top box (STB), a Personal Digital Assistant (PDA), a cellular telephone, a web appliance, a network router, switch or bridge, or any machine capable of executing instructions (sequential or otherwise) that specify actions to be taken by that machine. Further, while only a single machine 1100 is illustrated, the term “machine” shall also be taken to include any collection of machines that individually or jointly execute a set (or multiple sets) of instructions to perform any one or more of the methodologies discussed herein.

The example machine 1100 includes processing circuitry (e.g., the processor 1102, a CPU, a GPU, an ASIC, circuitry, such as one or more transistors, resistors, capacitors, inductors, diodes, logic gates, multiplexers, buffers, modulators, demodulators, radios (e.g., transmit or receive radios or transceivers), sensors 1121 (e.g., a transducer that converts one form of energy (e.g., light, heat, electrical, mechanical, or other energy) to another form of energy), or the like, or a combination thereof), a main memory 1104 and a static memory 1106, which communicate with each other via a bus 1108. The machine 1100 (e.g., computer system) may further include a video display unit 1110 (e.g., a liquid crystal display (LCD) or a cathode ray tube (CRT)). The machine 1100 also includes an alphanumeric input device 1112 (e.g., a keyboard), a user interface (UI) navigation device 1114 (e.g., a mouse), a disk drive or mass storage unit 1116, a signal generation device 1118 (e.g., a speaker), and a network interface device 1120.

The disk drive or mass storage unit 1116 includes a machine-readable medium 1122 on which is stored one or more sets of data structures and instructions (e.g., software) 1124 embodying or utilized by any one or more of the methodologies or functions described herein. The instructions 1124 may also reside, completely or at least partially, within the main memory 1104 and/or within the processor 1102 during execution thereof by the machine 1100, the main memory 1104 and the processor 1102 also constituting machine-readable media.

The machine 1100 as illustrated includes an output controller 1128. The output controller 1128 manages data flow to/from the machine 1100. The output controller 1128 is sometimes called a device controller, with software that directly interacts with the output controller 1128 being called a device driver.

While the machine-readable medium 1122 is shown in an embodiment to be a single medium, the term “machine-readable medium” may include a single medium or multiple media (e.g., a centralized or distributed database, and/or associated caches and servers) that store the one or more instructions 1124 or data structures. The term “machine-readable medium” shall also be taken to include any tangible medium that is capable of storing, encoding or carrying instructions for execution by the machine and that cause the machine to perform any one or more of the methodologies of the present disclosure, or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including by way of example semiconductor memory devices, e.g., Erasable Programmable Read-Only Memory (EPROM), EEPROM, and flash memory devices; magnetic disks such as internal hard disks and removable disks; magneto-optical disks; and CD-ROM and DVD-ROM disks.

The instructions 1124 may further be transmitted or received over a communications network 1126 using a transmission medium. The instructions 1124 may be transmitted using the network interface device 1120 and any one of a number of well-known transfer protocols (e.g., HTTP). Examples of communication networks include a LAN, a WAN, the Internet, mobile telephone networks, Plain Old Telephone (POTS) networks, and wireless data networks (e.g., WiFi and WiMax networks). The term “transmission medium” shall be taken to include any intangible medium that is capable of storing, encoding or carrying instructions for execution by the machine, and includes digital or analog communications signals or other intangible media to facilitate communication of such software.

As used herein, “communicatively coupled between” means that the entities on either of the coupling must communicate through an item therebetween and that those entities cannot communicate with each other without communicating through the item.

### Additional Notes

The above detailed description includes references to the accompanying drawings, which form a part of the detailed description. The drawings show, by way of illustration but not by way of limitation, specific embodiments in which the disclosure can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a,” “an,” “the,” and “said” are used when introducing elements of aspects of the disclosure or in the embodiments thereof, as is common in patent documents, to include one or more than one or more of the elements, independent of any other instances or usages of “at least one” or “one or more.” In this document, the term “or” is used to refer to a nonexclusive or, such that “A or B” includes “A but not B,” “B but not A,” and “A and B,” unless otherwise indicated.

In the appended claims, the terms “including” and “in which” are used as the plain-English equivalents of the respective terms “comprising” and “wherein.” Also, in the following claims, the terms “comprising,” “including,” and “having” are intended to be open-ended to mean that there may be additional elements other than the listed elements, such that elements after such a term (e.g., comprising, including, having) in a claim are still deemed to fall within the scope of that claim. Moreover, in the following claims, the terms “first,” “second,” and “third,” and so forth, are used merely as labels, and are not intended to impose numerical requirements on their objects.

Embodiments of the disclosure may be implemented with computer-executable instructions. The computer-executable instructions (e.g., software code) may be organized into one or more computer-executable components or modules. Aspects of the disclosure may be implemented with any number and organization of such components or modules. For example, aspects of the disclosure are not limited to the specific computer-executable instructions or the specific components or modules illustrated in the figures and described herein. Other embodiments of the disclosure may include different computer-executable instructions or components having more or less functionality than illustrated and described herein.

Method examples (e.g., operations and functions) described herein can be machine or computer-implemented at least in part (e.g., implemented as software code or instructions). Some examples can include a computer-readable medium or machine-readable medium encoded with instructions operable to configure an electronic device to perform methods as described in the above examples. An implementation of such methods can include software code, such as microcode, assembly language code, a higher-level language code, or the like (e.g., “source code”). Such software code can include computer-readable instructions for performing various methods (e.g., “object” or “executable code”). The software code may form portions of computer program products. Software implementations of the embodiments described herein may be provided via an article of manufacture with the code or instructions stored thereon, or via a method of operating a communication interface to send data via a communication interface (e.g., wirelessly, over the interne, via satellite communications, and the like).

Further, the software code may be tangibly stored on one or more volatile or non-volatile computer-readable storage media during execution or at other times. These computer-readable storage media may include any mechanism that stores information in a form accessible by a machine (e.g., computing device, electronic system, and the like), such as, but are not limited to, floppy disks, hard disks, removable magnetic disks, any form of magnetic disk storage media, CD-ROMS, magnetic-optical disks, removable optical disks (e.g., compact disks and digital video disks), flash memory devices, magnetic cassettes, memory cards or sticks (e.g., secure digital cards), RAMS (e.g., CMOS RAM and the like), recordable/non-recordable media (e.g., ROMs), EPROMS, EEPROMS, or any type of media suitable for storing electronic instructions, and the like. Such computer-readable storage medium coupled to a computer system bus may be accessible by the processor and other parts of the OIS.

In an embodiment, the computer-readable storage medium may have encoded a data structure for a treatment planning, wherein the treatment plan may be adaptive. The data structure for the computer-readable storage medium may be at least one of a Digital Imaging and Communications in Medicine (DICOM) format, an extended DICOM format, an XML format, and the like. DICOM is an international communications standard that defines the format used to transfer medical image-related data between various types of medical equipment. DICOM RT refers to the communication standards that are specific to radiation therapy.

In various embodiments of the disclosure, the method of creating a component or module can be implemented in software, hardware, or a combination thereof. The methods provided by various embodiments of the present disclosure, for example, can be implemented in software by using standard programming languages such as, for example, Compute Unified Device Architecture (CUDA), C, C++, Java, Python, and the like; and using standard machine learning/deep learning library (or API), such as tensorflow, torch and the like; and combinations thereof. As used herein, the terms “software” and “firmware” are interchangeable, and include any computer program stored in memory for execution by a computer.

A communication interface includes any mechanism that interfaces to any of a hardwired, wireless, optical, and the like, medium to communicate to another device, such as a memory bus interface, a processor bus interface, an Internet connection, a disk controller, and the like. The communication interface can be configured by providing configuration parameters and/or sending signals to prepare the communication interface to provide a data signal describing the software content. The communication interface can be accessed via one or more commands or signals sent to the communication interface.

The present disclosure also relates to a system for performing the operations herein. This system may be specially constructed for the required purposes, or it may comprise a general-purpose computer selectively activated or reconfigured by a computer program stored in the computer. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

In view of the above, it will be seen that the several objects of the disclosure are achieved, and other beneficial results attained. Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

The above description is intended to be illustrative, and not restrictive. For example, the above-described examples (or one or more aspects thereof) may be used in combination with each other. In addition, many modifications may be made to adapt a particular situation or material to the teachings of the disclosure without departing from its scope. While the dimensions, types of materials and coatings described herein are intended to define the parameters of the disclosure, they are by no means limiting and are example embodiments. Many other embodiments will be apparent to those of skill in the art upon reviewing the above description. The scope of the disclosure should, therefore, be determined with reference to the appended claims, along with the full scope of equivalents to which such claims are entitled.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the disclosure should be determined with reference to the appended claims, along with the full scope of equivalents to which such claims are entitled. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

1. A computer-implemented radiation treatment planning method, the method comprising:
obtaining intrabody imaging data and surface imaging data for determining a radiation treatment plan including a plurality of radiation treatment fraction sessions;
using the intrabody imaging data and the surface imaging data, generating a model relating 1) the intrabody imaging data having a three-dimensional (3D) patient representation to 2) a two-dimensional (2D) surface patient representation;
obtaining surface camera imaging data during a particular treatment fraction session; and
using the surface camera imaging data obtained during the particular treatment fraction session and the model, calculating a 3D patient representation during the particular treatment fraction session.

2. The method of claim 1, comprising modifying the radiation treatment plan for the particular treatment fraction session during that particular treatment fraction session.

3. The method of claim 1, wherein obtaining intrabody imaging data and surface imaging data for determining a radiation treatment plan including a plurality of radiation treatment fraction sessions includes using surface imaging data generated from the intrabody imaging data.

4. The method of claim 1, wherein obtaining intrabody imaging data and surface imaging data for determining a radiation treatment plan including a plurality of radiation treatment fraction sessions includes using surface imaging data generated from a surface camera.

5. The method of claim 1, wherein obtaining intrabody imaging data and surface imaging data is carried out during a treatment fraction session before initiating delivery of radiation therapy during that treatment fraction session.

6. The method of claim 5, wherein obtaining intrabody imaging data comprises obtaining computed tomography (CT) imaging data during a treatment fraction session prior to delivery of radiation therapy or obtaining cone-beam CT (CBCT) imaging during a treatment fraction session prior to delivery of radiation therapy.

7. The method of claim 1, comprising binning projections from intrabody imaging data to create a 4D image.

8. The method of claim 7, wherein the 4D image provides a 3D image over various respiratory phases.

9. The method of claim 8, comprising:
determining a reference external surface representation corresponding to a reference respiratory phase bin;
determining a first deformation vector field (DVF) between various respiratory phase bins and the reference respiratory phase bin; and
determining a second DVF corresponding to an external surface associated with the first DVF.

10. The method of claim 9, comprising using a principal component analysis (PCA) to generate a model using at least one of the first DVF or the second DVF.

11. The method of claim 10, comprising generating, using the model, an intrafractional intrabody image representation of the patient at various times during a particular radiation treatment fraction session.

12. The method of claim 11, comprising:
determining, using the model, at least one DVF during the particular radiation treatment fraction session.

13. The method of claim 11, comprising using the generated intrafractional intrabody image representation of the patient to modify a radiation treatment parameter during that particular radiation treatment fraction session.

14. The method of claim 13, wherein modifying the radiation treatment parameter during that particular radiation treatment fraction session is to accurate localization and tracking of a tumor.

15. The method of claim 1, comprising:
acquiring a 4D CBCT image during a particular radiation treatment fraction session;
generating a synthetic 4D CT image representation from the 4D CBCT image; and
generating an intrafractional intrabody image representation of the patient at various times during a particular radiation treatment fraction session using the synthetic 4D CT image representation.

16. The method of claim 15, comprising using the generated intrafractional intrabody image representation of the patient to modify at least one parameter during that particular radiation treatment fraction session.

17. A radiation treatment system configured to perform the method of claim 1.

18. A tangible or non-tangible computer readable medium encoded with instructions that, when executed by a processor, cause the processor to perform the method of claim 1.

19. A computer-implemented radiation treatment planning method, the method comprising:
obtaining pre-treatment CT imaging data for determining a radiation treatment plan including a plurality of radiation treatment fraction sessions;
obtaining CBCT imaging data during a treatment fraction session;
obtaining surface camera imaging data during the treatment fraction session; and
generating synthetic CT (sCT) imaging data from the CBCT imaging data and the surface camera imaging data.

20. The method of claim 19, wherein the generating sCT imaging data includes determining a deformation field vector (DVF) between the CT and CBCT imaging data.

21. The method of claim 20, wherein determining the deformation field vector (DVF) between the CT and CBCT imaging data includes using one or more limitations.

22. The method of claim 21, wherein determining the deformation field vector (DVF) between the CT and CBCT imaging data includes using one or more limitations from the surface camera imaging data.

23. The method of claim 21, wherein determining the deformation field vector (DVF) between the CT and CBCT imaging data includes using one or more limitations that one or more points on external contours match.

24. The method of claim 19, comprising modifying a radiation treatment plan using information derived from the DVF obtained during a particular treatment fraction session to modify a radiation treatment plan for that particular treatment fraction session or a later treatment fraction session.

25. The method of claim 19, comprising binning projections from the CBCT imaging data to create a 4D image.

26. The method of claim 25, wherein the 4D image provides a 3D image over various respiratory phases.

27. A radiation treatment system configured to perform the method of claim 19.

28. A tangible or non-tangible computer readable medium encoded with instructions that, when executed by a processor, cause the processor to perform the method of claim 19.
